(19) United States
(12) Patent Application Publication
(10) Pub. No.: US 2022/0010325 A1
(43) Pub. Date: Jan. 13, 2022

(54) QUANTITATIVE TRAIT LOCI (QTL) ASSOCIATED WITH SHATTERING-RESISTANT CAPSULES IN SESAME AND USES THEREOF

(71) Applicant: EQUI-NOM LTD., Kibbutz Givat Brenner (IL)

(72) Inventors: Oron GAR, Kibbutz Sde Yoav (IL); Arie ZACKAY, Jerusalem (IL); Gil SHALEV, Ramot Meir (IL)

(73) Assignee: EQUI-NOM LTD., Kibbutz Givat Brenner (IL)

(21) Appl. No.: 17/484,629

(22) Filed: Sep. 24, 2021

Related U.S. Application Data
(63) Continuation-in-part of application No. 16/706,752, filed on Dec. 8, 2019.
Application No. 16/706,752 is a continuation-in-part of application No. 16/109,346, filed on Aug. 22, 2018, now Pat. No. 10,577,623.
Application No. 16/109,346 is a continuation of application No. PCT/IL2018/050520, filed on May 14, 2018.
(60) Provisional application No. 62/506,397, filed on May 15, 2017.

(51) Int. Cl. C12N 15/82; C07K 14/415

(52) U.S. Cl. CPC C12N 15/8262; C07K 14/415

(57) ABSTRACT

Sesame plants with shattering-resistant capsules. and parts thereof are provided. Phenotypic and genotypic analysis of many sesame varieties was performed to derive markers for phenotypic traits that contribute to shattering-resistance, and a breeding simulation was used to identify the most common and most stable markers. Following verification of trait stability over several generations, markers and marker cassettes were defined as being uniquely present in the developed sesame lines. The resulting shatter-resistant sesame lines can be used to increase sesame productivity for its various uses.

### CROSS REFERENCE TO RELATED APPLICATIONS

This application is a continuation-in-part of U.S. patent application Ser. No. 16/706,752, filed Dec. 8, 2019, which is a continuation-in-part of U.S. patent application Ser. No. 16/109,346, filed Aug. 22, 2018, now U.S. Pat. No. 10,577,623, which is a continuation of PCT International Patent Application PCT/IL2018/050520, filed May 14, 2018, which claims the benefit of U.S. Provisional Patent Application 62/506,397, filed May 15, 2017, all of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Sep. 24, 2021, is named P-567291-US2_ST25.txt and is 9,116 bytes in size.

### FIELD OF THE INVENTION

The invention is in the field of sesame genetics and breeding. Specifically, the invention relates to genetic improvement for shattering-resistant capsules. More specifically, the invention relates to quantitative trait loci (QTL) conferring shattering-resistant capsules, and methods thereof, including methods for introgressing the QTL into elite germplasm in a breeding program for shattering-resistant capsules.

### BACKGROUND OF THE INVENTION

Sesame (Sesamum indicum) is an annual plant of the Pedaliaceae family, grown widely in tropical and subtropical areas and has a small (˜354 MB) diploid (2n=26) genome. Although sesame is considered to be one of the important and oldest of the oilseed plants, as it has been under cultivation in Asia for over 5000 years, sesame is a crop of developing countries due to its shattering capsules where the crop must be harvested manually for preventing losing the seeds. Thus, it requires intensive manual labor. The first and foremost obstacle to complete mechanization of harvesting is the dehiscence nature of its capsules. Even though some breeding efforts were done during the last seventy years in order to solve it through single gene mutations (ID, GS) and even a combination of few genes (ND and. IND varieties), still the majority of the world's sesame (over 99%) is dehiscent (shattering) type.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a sesame plant having shatter-resistant capsules, progeny thereof and/or part(s) thereof, wherein the sesame plant comprises at least three shattering-resistant capsule quantitative trait loci (QTLs) having corresponding nucleic acid genetic markers that are associated with seed retention phenotypic traits of the sesame plant, wherein the QTLs are combined in the sesame plant from a plurality of sesame varieties by computationally supported breeding, and comprise at least three of: QTL 1 with corresponding marker set forth in SEQ ID NO: 1 or 9, wherein the sesame plant is homozygous with respect to SEQ ID NO: 1 or heterozygous at QTL 1, QTL 2 with corresponding marker set forth in SEQ ID NO: 2 or 10, wherein the sesame plant is homozygous with respect to SEQ II) NO: 2 or heterozygous at QTL 2, QTL 3 with corresponding marker set forth in SEQ ID NO: 3 or 11, wherein the sesame plant is homozygous with respect to SEQ ID NO: 11 or heterozygous at QTL 3, QTL 4 with corresponding marker set forth in SEQ ID NO: 4 or 12, wherein the sesame plant is homozygous with respect to SEQ ID NO: 4 or heterozygous at QTL 4, QTL 5 with corresponding marker set forth in SEQ ID NO: 5 or 13, wherein the sesame plant is homozygous with respect to SEQ ID NO: 5 or heterozygous at QTL 5, QTL 6 with corresponding marker set forth in SEQ ID NO: 6 or 14, wherein the sesame plant is homozygous with respect to SEQ NO: 14 or heterozygous at QTL 6, QTL 7 with corresponding marker set forth in SEQ ID NO: 7 or 15, wherein the sesame plant is homozygous with respect to SEQ ID NO: 15 or heterozygous at QTL 7, and QTL 8 with corresponding marker set forth in SEQ ID NO: 8 or 16, wherein the sesame plant is homozygous with respect to SEQ ID NO: 16 or heterozygous at QTL 8.

One aspect of the present invention provides a sesame plant having shatter-resistant capsules, progeny thereof and/or part(s) thereof, wherein the sesame plant comprises a plurality of shattering-resistant capsule quantitative trait loci (QTLs) having corresponding nucleic acid genetic markers that are associated with seed retention phenotypic traits of the sesame plant, wherein the QTLs are combined in the sesame plant from a plurality of sesame varieties by computationally supported breeding, and comprise: QTL 1 with corresponding marker set forth in SEQ ID NO: 1 or 9, wherein the sesame plant is homozygous with respect to SEQ ID NO: 1 or heterozygous at QTL 1, QTL 2 with corresponding marker set forth in SEQ ID NO: 2 or 10, wherein the sesame plant is homozygous with respect to SEQ ID NO: 2 or heterozygous at QTL 2, QTL 6 with corresponding marker set forth in SEQ ID NO: 6 or 14, wherein the sesame plant is homozygous with respect to SEQ ID NO: 14 or heterozygous at QTL 6, and QTL 7 with corresponding marker set forth in SEQ ID NO: 7 or 15, wherein the sesame plant is homozygous with respect to SEQ ID NO: 15 or heterozygous at QTL 7.

One aspect of the present invention provides a sesame plant having shatter-resistant capsules, progeny thereof and/or part(s) thereof, wherein the sesame plant comprises a plurality of shattering-resistant capsule quantitative trait loci (QTLs) having corresponding nucleic acid genetic markers that are associated with seed retention phenotypic traits of the sesame plant, wherein the QTLs are combined in the sesame plant from a plurality of sesame varieties by raputationally supported breeding, and comprise: QTL 1 with corresponding marker set forth in SEQ ID NO: 1 or 9, wherein the sesame plant is homozygous with respect to SEQ ID NO: 1 or heterozygous at QTL1, QTL 4 with corresponding marker set forth in SEQ ID NO: 4 or 12, wherein the sesame plant is homozygous with respect to SEQ ID NO: 4 or heterozygous at QTL 4, and QTL 7 with corresponding marker set forth in SEQ ID NO: 7 or 15, wherein the sesame plant is homozygous with respect to SEQ ID NO: 15 or heterozygous at QTL 7.

Advantageously, disclosed embodiments provide improved sesame lines and breeding methods that yield shattering-resistant capsules (e.g., plants with capsules that provide increased resistance to shattering).

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout. In the accompanying drawings:

FIGS. 1A and 1B show QTLs and linked markers on Sesamum indicum linkage groups and chromosomes, respectively, according to some embodiments of the invention.

FIG. 2 is a high-level schematic illustration of computationally supported breeding methods, according to some embodiments of the invention.

FIG. 3 provides a non-limiting example of a genealogy of one of the disclosed sesame varieties, illustrating schematically the complex breeding process outlined herein, according to some embodiments of the invention.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Unless specifically stated otherwise, as apparent from the following discussions, it is appreciated that throughout the specification discussions utilizing terms such as “processing”, “computing”, “calculating”, “determining”, “enhancing”, “deriving” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a”, “an”, and “the” include the plural reference, and reference to a particular numerical value includes at least that particular value, unless the context clearly indicates otherwise. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

The term “quantitative trait locus” or “QTL” refers to a polymorphic genetic locus with at least two alleles that differentially affect the expression of a continuously distributed phenotypic trait.

The term “associated with” or “associated” in the context of this invention refers to, e.g., a nucleic acid and a phenotypic trait, that are in linkage disequilibrium, e.g., the nucleic acid and the trait are found together in progeny plants more often than if the nucleic acid and phenotype segregated separately.

The term “linkage disequilibrium” refers to a non-random segregation of genetic loci. This implies that such loci are in sufficient physical proximity along a length of a chromosome that they tend to segregate together with greater than random frequency.

The term “genetically linked” refers to genetic loci that are in linkage disequilibrium and statistically determined not to assort independently. Genetically linked loci assort dependently from 51% to 99% of the time or any whole number value therebetween, preferably at least 60%, 70%, 80%, 90%, 95% or 99%.

The term “proximal” means genetically linked, typically within about 20 centimorgans (cM).

The term “marker” or “molecular marker” refers to a genetic locus (a “marker locus”) used as a point of reference when identifying genetically linked loci such as a QTL. The term also refers to nucleic acid sequences complementary to the genomic sequences, such as nucleic acids used as probes.

The term “interval” refers to a continuous linear span of chromosomal DNA with termini defined by and including molecular markers.

The terms “nucleic acid,” “polynucleotide,” “polynucleotide sequence” and “nucleic acid sequence” refer to single-stranded or double-stranded deoxyribonucleotide or ribonucleotide polymers, or chimeras thereof. As used herein, the term can additionally or alternatively include analogs of naturally occurring nucleotides having the essential nature of natural nucleotides in that they hybridize to single-stranded nucleic acids in a manner similar to naturally occurring nucleotides (e.g,, peptide nucleic acids). Unless otherwise indicated, a particular nucleic acid sequence of this invention optionally encompasses complementary sequences, in addition to the sequence explicitly indicated. The term “gene” is used to refer to, e.g., a cDNA and an mRNA encoded by the genomic sequence, as well as to that genomic sequence.

The term “homologous” refers to nucleic acid sequences that are derived from a common ancestral gene through natural or artificial processes (e.g., are members of the same gene family), and thus, typically, share sequence similarity. Typically, homologous nucleic acids have sufficient sequence identity that one of the sequences or its complement is able to selectively hybridize to the other under selective hybridization conditions. The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences have about at least 80% sequence identity, preferably at least 90% sequence identity, and most preferably 95%, 97%, 99%, or 100% sequence identity with each other. A nucleic acid that exhibits at least some degree of homology to a reference nucleic acid can be unique or identical to the reference nucleic acid or its complementary sequence.

The term “isolated” refers to material, such as a nucleic acid or a protein, which is substantially free from components that normally accompany or interact with it in its naturally occurring environment. The isolated material optionally comprises material not found with the material in its natural environment, e.g., a cell. In addition, if the material is in its natural environment, such as a cell, the material has been placed at a location in the cell (e.g., genome or subcellular organelle) not native to a material found in that environment. For example, a naturally occurring nucleic acid (e.g., a promoter) is considered to be isolated if it is introduced by non-naturally occurring means to a locus of the genome not native to that nucleic acid. Nucleic acids which are “isolated” as defined herein, are also referred to as “heterologous” nucleic acids.

The term “introduced” when referring to a heterologous or isolated nucleic acid refers to the incorporation of a nucleic acid into a eukaryotic or prokaryotic cell where the nucleic acid can be incorporated into the genome of the cell (e.g., chromosome, plasmid, plastid or mitochondrial DNA), converted into an autonomous replicon, or transiently expressed (e.g., transfected mRNA). The term includes such nucleic acid introduction means as “transfection,” “transformation” and “transduction.”

The term “host cell” means a cell which contains a heterologous nucleic acid, such as a vector, and supports the replication and/or expression of the nucleic acid. Host cells may be prokaryotic cells such as E. coli, or eukaryotic cells such as yeast, insect, amphibian, or mammalian cells. Preferably, host cells are monocotyledonous or dicotyledonous plant cells. In the content of the invention, one particularly preferred monocotyledonous host cell is a soybean host cell.

The term “dicot” refers to the subclass of angiosperm plants also knows as “Dicotyledoneae” and includes reference to whole plants, plant organs (e.g., leaves, stems, roots, etc.), seeds, plant cells, and progeny of the same. Plant cell, as used herein includes, without limitation, seeds, suspension cultures, embryos, meristematic regions, callus tissue, leaves, roots, shoots, gametophytes, sporophytes, pollen, and microspores.

The term “crossed” or “cross” in the context of this invention means the fusion of gametes via pollination to produce progeny (e.g., cells, seeds, or plants). The term encompasses both sexual crosses (the pollination of one plant by another) and selfing (self-pollination, e.g., when the pollen and ovule are from the same plant).

“Plant,” as used herein, refers broadly to the whole plant or any parts or derivatives thereof, such as plant organs (e.g., harvested or non-harvested storage organs, bulbs, tubers, fruits, leaves), plant cells, plant protoplasts, plant cell tissue cultures from which whole plants can be regenerated, plant calli, plant cell clumps, and plant cells that are intact in plants, or parts of plants, such as embryos, pollen, ovules, fruits (e.g., capsule, harvested tissues or organs), flowers. leaves, seeds, clonally propagated plants, roots, stems, root tips. Also, any developmental stage is included, such as seedlings, immature and mature plant parts.

“Variety,” as used herein, refers broadly to a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

“Phenotype,” as used herein, refers broadly to the observable external and/or physiological appearance of the plant as a result of the interaction between its genotype and its environment. It includes all observable morphological and physiological characteristics.

“Genotype,” as used herein, refers broadly to the total of inheritable genetic information of a plant, partly influenced by the environmental factors, which is expressed in the phenotype.

“Hybrid” or “hybrid plant,” as used herein, refers broadly to a plant produced by the inter-crossing (cross-fertilization) of at least two different plants or plants of different parent lines. The seeds of such a cross (hybrid seeds) are encompassed, as well as the hybrid plants grown from those seeds and plant parts derived from those grown plants (e.g., seeds).

“F1, F2, seq al.,” as used herein, refers broadly to the consecutive related generations following a cross between two parent plants or parent lines. The plants grown from the seeds produced by crossing two plants or lines is called the F1 generation. Selfing the F1 plants results in the F2 generation.

The terms “introgression” or “hybridization”, as used herein,refer to the transmission of a desired alleles of genetic loci from one genetic background to another by classical breeding approach. For example, introgression of a desired allele at a specified locus can be transmitted to at least one progeny plant via a sexual cross between two parent plants, where at least one of the parent, plants has the desired allele within its genome and due to recombination events in F2 population of a specific cross. The desired allele can be a selected allele/QTL represented by a specific genetic marker.

Disclosed embodiments provide QTLs that confer shattering-resistant capsules, and elite sesame varieties having shattering-resistant capsules. Shattering-resistant capsules were characterized in fully developed capsules having at most 10% seed moisture, and the shatter-resistant capsules were characterized by at least one of the following features: (i) at least 80% seed retention after shaking the plant, (ii) at least 80% seed retention after the capsules are turned upside down, (iii) a ratio of at least 5:1 between a total length of the capsule and a length of a zone in which the capsule tips are open, and/or 20-30% of the capsules retain 90-95% of the seeds in fully developed green capsules before drying.

Various embodiments comprise sesame plants with shatter-resistant capsules, or part(s) thereof, that comprise a plurality of loci associated with a corresponding plurality of quantitative trait loci (QTLs) having a corresponding plurality of nucleic acid genetic markers that are associated with a plurality of phenotypic traits of the sesame plant. The QTLs are combined in the sesame plants from a plurality of sesame varieties according to computationally supported breeding tools. Phenotypic and genotypic analyses of many sesame varieties were performed to derive markers for phenotypic traits that contribute to having shatter-resistant capsules, and a breeding simulation was used to identify the most common and most stable markers. Following verification of trait stability over several generations, markers and marker cassettes were defined as being uniquely present in the developed sesame lines. The resulting sesame lines with shatter-resistant capsules can be used to improve mechanized sesame production for its various uses.

It is noted that disclosed sesame plants with shatter-resistant capsules are hybridized in that none of the disclosed varieties occurs in nature or in known worldwide sesame varieties. The sesame plants with shatter-resistant capsules are characterized by the disclosed QTL markers which were judiciously detected in other varieties, selected and gradually introduced in the disclosed combinations to yield the disclosed sesame plants with shatter-resistant capsules. Once specific disclosed sesame plants with shatter-resistant capsules were achieved, further breeding was used to stabilize the varieties and assure constant phenotypes for sesame production, making the varieties pure lines. The term “hybridized” is used herein to define disclosed varieties having QTL markers and traits collected during the breeding process from different varieties that were determined and hybridized during the highly complicated computationally-supported breeding methods described below, in which the genotypes of multiple sesame varieties have been judiciously combined and analyzed, to discover and accumulate the recited QTL markers and corresponding phenotypical traits into the disclosed sesame plant with shatter-resistant capsules. Although the recited sesame plants are not genetically modified by sequences originating from other species, they cannot he reached merely by spontaneous natural reproductive events, as is evident by the detailed and intentional breeding program that was applied to specifically measure required characteristics, detect. corresponding markers using bioinformatics methods and combine the detected QTLs in the selected varieties by classic breeding approaches (e.g., hand pollination crosses and single plant selections). For example, any further generation derived from the disclosed sesame plants with shatter-resistant capsules is also understood to be a sesame plant with shatter-resistant capsules.

In the following description, two combinations of unique QTLs, referred to as QTL cassettes, were detected to differentiate disclosed breeding material with shattering resistant capsules from worldwide sesame lines. The cassettes' discovery was based on ca. 120 accessions derived from germplasm included five hundreds different sesame lines that were obtained from the U.S. National Plant Germplasm System (NPGC) and courtesy of Prof. Amram Ashri's sesame germplasm collection (Ashri, 1998) and a set of ca. 250 propriety lines. Following the cassettes discovery, the disclosed germplasm was tested with respect to 250 world accessions to demonstrate their uniqueness, as being clearly distinct from known sesame varieties.

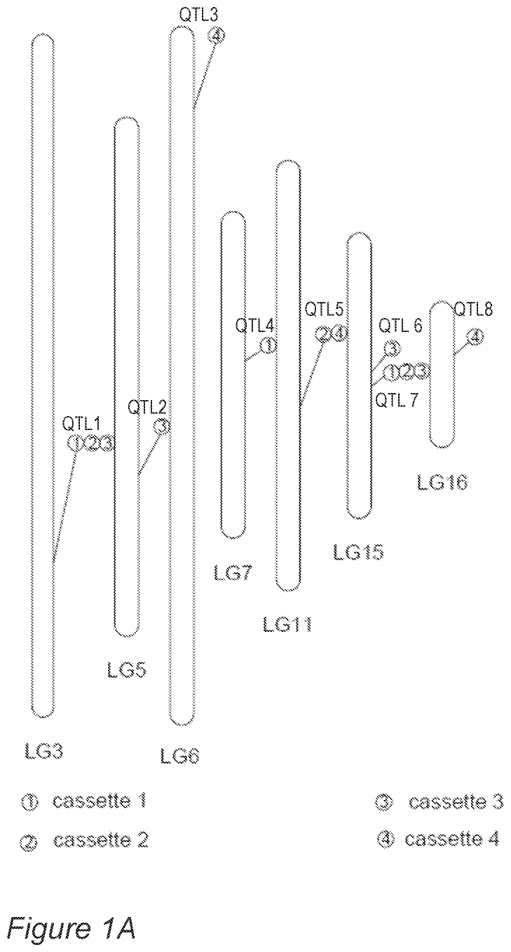

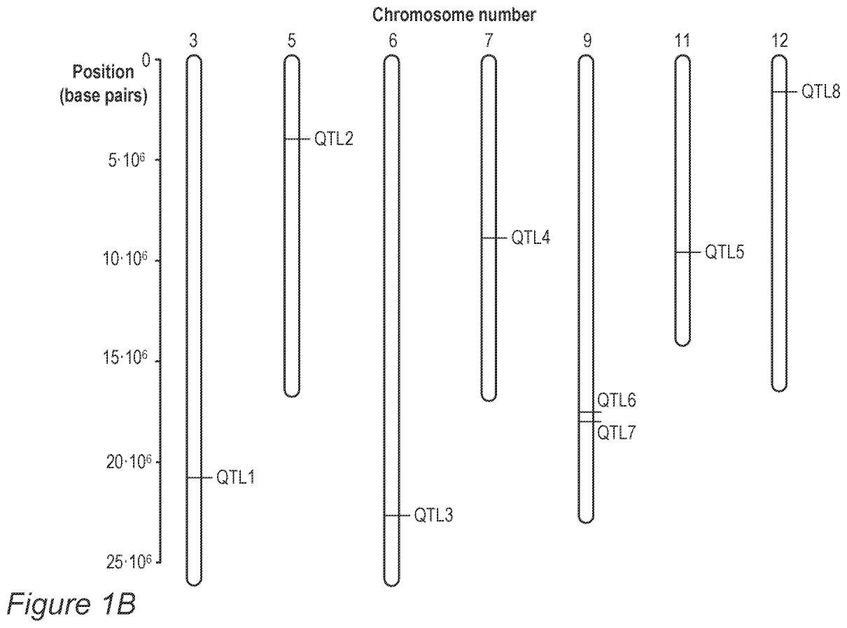

FIGS. 1A and 1B show QTLs and linked markers on Sesamum indicum linkage groups according to the first version of the sesame genome, and on Sesamum indicum chromosomes according to the second version of the sesame genome, respectively, according to some embodiments of the invention. In FIG. 1A, the circles with numbers represent marker combination sets (“cassettes”). Circles with the numbers 1, 2, 3, and 4, represent cassettes 1, 2, 3, and 4, respectively. In FIG. 1B only the QTLs are illustrated, with reference to Table 2 below, indicating the combinations of QTLs into the respective cassettes. It is noted that the actual QTLs and markers are the same, with only their mapping being different in the two versions of the genome.

The QTLs of the invention comprise one or more of QTLs 1 to 8. In one embodiment, the alleles of one or more markers linked to QTLs 1-8 are homozygous. In another embodiment, the alleles of one or more markers linked to QTLs 1-8 are heterozygous.

“QTL 1,” as used herein refers to a polymorphic genetic locus linked to genetic marker LG3_19205572 in sesame linkage group 3. In one embodiment, the alleles of LG3_19205572 are homozygous. In another embodiment, the alleles of LG3_19205572 are heterozygous. In one embodiment, a first allele of LG3_19205572 may have the base ‘C.’ at position 19205572, and a second allele may have the base ‘T’ instead of ‘C’ at position 19205572. The nucleic acid sequence of the first allele of LG3_9205572 marker is set forth in SEQ ID NO: 1, and the nucleic acid sequence of the second allele of LG3_19205572 marker is set forth in SEQ ID NO: 9. In certain embodiments, the sesame plant is homozygous with respect to SEQ ID NO: 1 or heterozygous at QTL 1. The sequences described herein with respect to their linkage groups (LGs) are from Sesame genome version 1 (Wang et at., Genome Biology, 2014, 15(2): R39, which is incorporated herein in its entirety, and see FIG. 1A), while same sequences are also referred to with respect to their chromosomal position from the Genome V2.0 version (Wang et al. 2016, Updated sesame genome assembly and fine mapping of plant height and seed coat color QTLs using a new high-density, BMC Genomics 17:31, which is incorporated herein in its entirety, and see FIG. 1B), In the Genome V2.0 version, the marker for QTL 1 is on chromosome 3 (+, reference strand) at position 20792058.

SEQ ID NO: 1 (SNP base bold and spaced and spaced):
cctttagcag ggcatcactc tcttcaacat cgtactgcac
cgagaggaat ttcgaagtga gaaaaacttg gggctccgac
acttcccctt tgctgtcgtt ttgaaaagtg agcttcaatt
tcgccaaatt atccaacaat ttgcacgaga c tttattaaa
gaacacggag ctctcgctgt cgaactccga cgtaactcgc
agcttcggcc gacccatttg gaagagcccc agaaaaccgc
cgcctttcga accgccgtcg tccacggcgg gggatggtgg
tggagacaga taatagggtt g
SEQ ID NO: 9 (SNP base bold and spaced and spaced):
cctttagcag ggcatcactc tcttcaacat cgtactgcac
cgagaggaat ttcgaagtga gaaaaacttg gggctccgac
actteccett tgctgtcgtt ttgaaaagtg agcttcaatt
tcgccaaatt atccaacaat ttgcacgaga t tttattaaa
gaacacggag ctctegctgt cgaactccga cgtaactcgc
agcttcggcc gacccatttg gaagagcccc agaaaaccgc
cgcctttcga accgccgtcg tccacggcgg gggatggtgg
tggagacaga taatagggtt g

“QTL 2,” as used herein refers to a polymorphic genetic locus linked to genetic marker LG5_12832234 in sesame linkage group 5. In one embodiment, the alleles of LG5_12832234 are homozygous. In another embodiment, the alleles of LG5_12832234 are heterozygous. In one embodiment, a first allele of LG5_12832234 may have the base ‘C’ at position 12832234, and a second allele may have the base ‘T’ instead of ‘C’ at position 12832234. The nucleic acid sequence of the first allele of LG5_12832234 marker is set forth in SEQ ID NO: 2, and the nucleic acid sequence of the second allele of LG5_12832234 marker is set forth in SEQ ID NO: 10. In certain embodiments, the sesame plant is homozygous with respect to SEQ ID NO: 2 or heterozygous at QTL 2. In the Genome V2.0 version, the marker for QTL 2 is on chromosome 5 (−, reverse complement strand) at position 3973879.

SEQ ID NO: 2 (SNP base bold and spaced):
ccgggccaca actgatgctt attgtgttgc caaatatggt
cagaagtggg tcaggacaag gacaatcatc gacagttttg
ctcctaaatg gaatgagcag tatacttggg aagattttga
tccttgcact gttgtcacca ttggtgtatt c gataattgt
catctgcaag gtggagataa agctggaagg gattcaagaa
ttgggaaggt aagaattcgc ctttcaactc tggaaacaga
ccgtgtgtac actcattctt atcctcttct agttttgcat
ccttccgggg taaaaaaagat g
SEQ ID NO: 10 (SNP base bold and spaced):
ccgggccaca actgatgctt attgtgttgc caaatatggt
cagaagtggg tcaggacaag gacaatcatc gacagttttg
ctcctaaatg gaatgagcag tatacttggg aagtttttga
tccttgcact gttgtcacca ttggtgtatt t gataattgt
catctgcaag gtggagataa agctggaagg gattcaagaa
ttgggaaggt aagaattcgc ctttcaactc tggaaacaga
ccgtgtgtac actcattctt atcctcttct agttttgcat
ccttccgggg taaaaaagat g

“QTL 3,” as used herein refers to a polymorphic genetic locus linked to genetic marker LG6_2739268 in sesame linkage group 6. In one embodiment, the alleles of LG6_2739268 are homozygous. In another embodiment, the alleles LG6_2739268 are heterozygous. In one embodiment, a first allele of LG6_2739268 may have the base ‘T’ at position 2739268, and a second allele may have the base ‘C’ instead of ‘T’ at position 2739268. The nucleic acid sequence of the first allele of LG6_2739268 marker is set forth in SEQ ID NO: 3, and the nucleic acid sequence of the second allele of LG6_2739268 marker is set forth in SEQ ID NO: 11. In certain embodiments, the sesame plant is homozygous with respect to SEQ H) NO: 11 or heterozygous at QTL 3. In the Genome V2.0 version, the marker for QTL 3 is on chromosome 6 (−, reverse complement strand) at position 22739924.

SEQ ID NO: 3 (SNP base bold and spaced):
ttagggcgtc gaattacttg gacgcgccga gcaagctagc
ctgctgcaac tcaagctcgt tgagctgctg ctcgcggtcc
atctctataa tcagaggcac gacgagcacg aggaaggtgg
tgccggcaat ccacgcggct tttccggtgc t cttcaggag
cttctttgcg acgtaggcgg tatcaaaagc cgctttcttg
ccgcggtaca cgatgggtga ctgggaaacg gaggtggaaa
cacgggacag gattccgtcg tccgacgacg ctccgcctcc
tctagtagac attctggtga a
SEQ ID NO: 11 (SNP base bold and spaced):
ttagggcgtc gaattacttg gacgcgccga gcaagctagc
ctgctgcaac tcaagctcgt tgagctgctg ctcgcggtcc
atctctataa tcagaggcac gacgagcacg aggaaggtgg
tgccggcaat ccacgcggct tttccggtgc c cttcaggag
cttctttgcg acgtaggcgg tatcaaaagc cgctttcttg
ccgcggtaca cgatgggtga ctgggaaacg gaggtggaaa
cacgggacag gattccgtcg tccgacgacg ctccgcctcc
tctagtagac attctggtga a

“QTL 4,” as used herein refers to a polymorphic genetic locus linked to genetic marker LG7_5141423 in sesame linkage group 7. In one embodiment, the alleles of LG7_5141423 are homozygous. In another embodiment, the alleles LG7_5141423 are heterozygous. In one embodiment, a first allele of LG7 _5141423 may have the base ‘C’ at position 5141423, and a second allele may have the base ‘G’ instead of ‘C’ at position 5141423. The nucleic acid sequence of the first allele of LG7_5141423 marker is set forth in SEQ ID NO: 4, and the nucleic acid sequence of the second allele of LG7_5141423 marker is set forth in SEQ ID NO: 12. In certain embodimen(s, the sesame plant is homozygous with respect to SEQ ID NO: 4 or heterozygous at QTL 4. In the Genome V2.( )version, the marker for QTL 4 is on chromosome 7 (+, reference strand) at position 8855456.

SEQ ID NO: 4 (SNP base bold and spaced):
gccattgatt taccaaaagc accaacccat tttggtgaga
taattgggaa acttgttttg gctggagcct tggacttcaa
caaggtggca agggatattc tggcaaaagt aggtgacgac
tattaccaaa aggccatatt tactgctgct c tgaaggttg
tcagctctga tccttcagga
aaggcattgc tcgattcaca ggcgtctgat gtcgctgcct
gcgagagttt attttagagc tcactccttg ttatgggaat
tactggaaac atttgtaacc tcatagaaga aatgtgctat t
SEQ ID NO: 12 (SNP base bold and spaced):
gccattgatt taccaaaagc accaacccat tttggtgaga
taattgggaa acttgttttg gctggagcct tggacttcaa
caaggtggca agggatattc tggcaaaagt aggtgacgac
tattaccaaa aggccatatt tactgctgct g tgaaggttg
tcagctctga tccttcagga
aaggcattgc tcgattcaca ggcgtctgat gtcgctgcct
gcgagagttt attttagagc tcactccttg ttatgggaat
tactggaaac atttgtaacc tcatagaaga aatgtgctat t

“QTL 5,” as used herein refers to a polymorphic genetic locus linked to genetic marker LG11_8864255 in sesame linkage group 11. In one embodiment, the alleles of LG11_8864255 are homozygous. In another embodiment, the alleles LG11_8864255 are heterozygous. In one embodiment, a first allele of LG11 _8864255 may have the base ‘C’ at position 8864255, and a second allele may have the base ‘G’ instead of ‘C’ at position 8864255. The nucleic acid sequence of the first allele of LG11_8864255 marker is set forth in SEQ ID NO: 5, and the nucleic acid sequence of the second allele of LG11_8864255 marker is set forth in SEQ 13. In certain embodiments, the sesame plant is homozygous with respect to SEQ ID NO: 5 or heterozygous at QTL5, In the Genome V2.0 version, the marker for QTL 5 is on chromosome 11 (+, reference strand) at position 9582098.

SEQ ID NO: 5 (SNP base bold and spaced):
aatagaaaat tggtaattca aaagggaaag atggaaaaat
gattaccatc cctgtcttca gctgcctcaa cattcacaga
atccttcgca agcacatctg tctctgtgga tgctttactt
tttattaccg gaaactgaac attgtgccgt c catttttcc
taaaaagcat ataatctctc tttgacagtg ttttcattac
aagagttcct gcatttccat cattctaaga aagagaggtt
gattaaggca tccagcatcg cataaacata accaggaaaa
tcgggagcaa acatagtact g
SEQ ID NO: 13 (SNP base bold and spaced):
aatagaaaat tggtaattca aaagggaaag atggaaaaat
gattaccatc cctgtcttca gctgcctcaa cattcacaga
atccttcgca agcacatctg tctctgtgga tgctttactt
tttattaccg gaaactgaac attgtgccgt g catttttcc
taaaaagcat ataatctctc tttgacagtg ttttcattac
aagagttcct gcatttccat cattctaaga aagagaggtt
gattaaggca tccagcatcg cataaacata accaggaaa
a tcgggagcaa acatagtact g

“QTL 6,” as used herein refers to a polymorphic genetic locus linked to genetic marker LG15_4900868 in sesame linkage group 15. The alleles of LG15_4900868 may be homozygous with respect to SEQ ID NO: 14 or heterozygous. In one embodiment, a first allele of LG15_4900868 may have the base ‘G’ at position 4900868, and a second allele may have the base ‘A’ instead of ‘G’ at position 4900868. The nucleic acid sequence of the first allele of LG15_4900868 marker is set forth in SEQ ID NO: 6; the nucleic acid sequence of the second allele of LG15_4900868 marker is set forth in SEQ ID NO: 14. In certain embodiments, the sesame plant is homozygous with respect to SEQ ID NO: 14 or heterozygous at QTL 6. In the Genome V2.0 version, the marker for QTL 6 is on chromosome 9 (−, reverse complement strand) at position 17947299.

SEQ ID NO: 6 (SNP base bold and spaced):
ggaggcaaaa gaatacgggt tggttgatgc agtgatcgat
gatggcaagc ctggactagt cgcacccatc gcagatactg
cacccccacc aaaaacccgt gtctgggatc tttggaaaat
cgaaggcagt aaaaaagcca agaaaaactt g ccctccgaa
gagaaactat tacaaaatgg atacacagtt ggccaaggtg
aagatgacag aagcacggaa caggtagagg aagcaccaac
atctcaatga gtaatgaatg ttgagatatt tcttgtatac
actgtcaaac attgtagcta g
SEQ ID NO: 14 (SNP base bold and spaced):
ggaggcaaaa gaatacgggt tggttgatgc agtgatcgat
gatggcaagc ctggactagt cgcacccatc gcagatactg
cacccccacc aaaaacccgt gtctgggatc tttggaaaat
cgaaggcagt aaaaaagcca agaaaaactt a ccctccgaa
gagaaactat tacaaaatgg atacacagtt ggccaaggtg
aagatgacag aagcacggaa caggtagagg aagcaccaac
atctcaatga gtaatgaatg ttgagatatt tcttgtatac
actgtcaaac attgtagcta g

“QTL 7,” as used herein refers to a polymorphic genetic locus linked to genetic marker LG15_5315334 in sesame linkage group 15. The alleles of LG15_5315334 may be homozygous with respect to SEQ ID NO: 15 or heterozygous. In one embodiment, a first allele of LG15_5315334 may have the base ‘T’ at position 5315334, and a second allele may have the base ‘C’ instead of ‘T’ at position 5315334. The nucleic acid sequence of the first allele of LG15_5315334 marker is set forth in SEQ ID NO: 7, and the nucleic acid sequence of the second allele of LG15_5315334 marker is set forth in SEQ ID NO: 15. In certain embodiments, the sesame plant is homozygous with respect to SEQ ID NO: 15 or heterozygous at QTL 7. In the Genome V2.0 version, the marker for QTL 7 is on chromosome 9 (−, reverse complement strand) at position 17532833.

SEQ ID NO: 7 (SNP base bold and spaced):
agttgataaa ctgttgacta atcaaataat acgcattctg
cacgcactca caaatactat gattgttgtt tactgaataa
ggttttcatg gaattttcac aggttaaatt ctagtaatca
cataaaagta tgtcgccagc tgactcttca t gcgaggaaa
atgtgtacat ggccaagttg
gccgaacagg ctgagaggta tgaggagatg gttgaattca
tggagaaggt tgtgaaggcc gtggacactg atgagctgac
agtcgaggaa aggaaccttc tctctgtggc atacaagaat g
SEQ ID NO: 15 (SNP base bold and spaced):
agttgataaa ctgttgacta atcaaataat acgcattctg
cacgcactca caaatactat gattgttgtt tactgaataa
ggttttcatg gaattttcac aggttaaatt ctagtaatca
cataaaagta tgtcgccagc tgactcttca c gcgaggaaa
atgtgtacat ggccaagttg gccgaacagg ctgagaggta
tgaggagatg gttgaattca tggagaaggt tgtgaaggcc
gtggacactg atgagctgac agtcgaggaa aggaaccttc
tctctgtggc atacaagaat g

“QTL 8,” as used herein refers to a polymorphic genetic locus linked to genetic marker LG16_1563304 in sesame linkage group 16. In one embodiment, the alleles of LG11_8864255 are homozygous. In another embodiment, the alleles LG16_1563304 are heterozygous. In one embodiment, a first allele of LF16_1563304 may have the base ‘A’ at position 1563304, and a second allele may have the base ‘G’ instead or ‘A’ at position 1563304. The nucleic acid sequence of the first allele of LG16_1563304 marker is set forth in SEQ ID NO: 8, and the nucleic acid sequence of the second allele of LG16_1563304 marker is set forth in SEQ ID NO: 16. In certain embodiments, the sesame plant is homozygous with respect to SEQ ID NO: 16 or heterozygous at QTL 8, In the Genome V2.0 version, the marker for QTL 8 is on chromosome 12 (+, reference strand) at position 1563304.

SEQ ID NO: 8 (SNP base bold and spaced):
acgtaaatgt ttggatttaa tgtaatttaa tctaatgata
ttgcaaatga gtaaattact cccaaattat tcggataaag
caatttaact tttggtttct tgtgagataa cattgcatgt
cctttatgaa ccaagagcag tggccgaggg a ctggtggtg
gtaccgttgc caaggatgca
ttaggcaatg atgttattgc agcggaatgg ctcaaaaacc
atggacctgg cgatcggaca cttacacagg ggctgaaggt
aattattgat ctagttgcaa aatagatcac ttattggctt t
SEQ ID NO: 16 (SNP base bold and spaced):
acgtaaatgt ttggatttaa tgtaatttaa tctaatgata
ttgcaaatga gtaaattact cccaaattat tcggataaag
caatttaact tttggtttct tgtgagataa cattgcatgt
cctttatgaa ccaagagcag tggccgaggg g ctggtggtg
gtaccgttgc caaggatgca ttaggcaatg atgttattgc
agcggaatgg ctcaaaaacc atggacctgg cgatcggaca
cttacacagg ggctgaaggt aattattgat ctagttgcaa
aatagatcac ttattggctt t

In one aspect, the invention provides a combination of markers wherein the combination comprises LG3_19205572, LG5_12832234, LG6_2739268, LG7_5141423, LG11_8864255, LG15_4900868, LG15_5315334, and LG16_1563304, and wherein the alleles for LG3_19205572, LG7_5141423, and LG15_5315334 are heterozygous.

In another aspect, the invention provides a combination of markers wherein the combination comprises LG3_19205572, LG5_12832234, LG6_2739268, LG7_5141423, LG11_8864255, LG15_4900868, LG15_5315334, and LG16_1563304, and wherein the alleles for LG3_19205572, LG11_8864255, and LG15_5315334 are heterozygous.

In another aspect, the invention provides a combination of markers wherein the combination comprises LG3_19205572, LG5_12832234, LG6_2739268, LG7_5141423, LG11_8864255, LG15_4900868, L015_5315334, and LG16_1563304, and wherein the alleles for LG3_19205572, LG5_12832234, LG15_4900868, and LG15_5315334 are heterozygous.

In another aspect, the invention provides a combination of markers wherein the combination comprises LG3_19205572, LG5__12832234, LG6_2739268, LG7_5141423, LG11_8864255, LG15_4900868, LG15_5315334, and LG16_1563304, and wherein the alleles for LG6_2739268, LG11_8864255, and LG16_1563304 are heterozygous.

Suitable markers are genetically linked to the QTLs identified herein as associated with shattering-resistant capsules, and are within the scope of the present invention. Markers can be identified by any of a variety of genetic or physical mapping techniques. Methods of determining whether markers are genetically linked to a QTL (or to a specified marker) associated with shattering-resistant capsules are known to those of skill in the art and include, for example, but are not limited to, interval mapping (Lander and Botstein (1989) Genetics 121:185), regression mapping (Haley and Knott (1992) Heredity 69:315) or MQM mapping (Jansen (1994) Genetics 138:871). In addition, physical mapping techniques such as, for example, chromosome walking, contig mapping and assembly, and the like, can be employed to identify and isolate additional sequences useful as markers in the context of the present invention.

In another embodiment, the markers are homologous markers. Homologous markers can be identified by, for example, selective hybridization to a reference sequence. The reference sequence is typically a unique sequence, such as, for example, unique oligonucleotide primer sequences, ESTs, amplified fragments (e.g., corresponding to amplified fragment length polymorphisms (AFLP) markers) and the like, derived from the marker loci of the invention.

In one example, the homologous markers hybridize with their complementary region. For example, two single-stranded nucleic acids “hybridize” when they form a double-stranded duplex. The double stranded region can include the full-length of one or both of the single-stranded nucleic acids, or all of one single stranded nucleic acid and a subsequence of the other single-stranded nucleic acid, or the double stranded region can include a subsequence of each nucleic acid. Selective hybridization conditions distinguish between nucleic acids that are related, e.g., share significant sequence identity with the reference sequence (or its complement) and those that associate with the reference sequence in a non-specific manner. Generally, selective hybridization conditions are known in the art.

The methods for detecting genetic markers are known in the art and fully described, for example, in U.S. Pat. Nos. 8,779,233; 6,670,524; 8,692,064; 9,000,258; 8,987,549; 8,637,729; 6,455,758; 5,981,832; 5,492,547; 9,167,795; 8,656,692; 8,664,472; 8,993,835; 9,125,372; 9,144,220; 9,462,820; 7,250,552.; and 9,485,936; and U.S. Patent Application Publications Nos. 2015/0082476; 2011/0154528; 2014/0215657; 2017/0055481; 2015/0150155; and 2015/0101073, each of which is incorporated by reference herein in its entirety.

Markers corresponding to genetic polymorphisms between members of a population can be detected by numerous methods, described in the art, for example, include but are not limited to, restriction fragment length polymorphisms, isozyme markers, allele specific hybridization (ASH), amplified variable sequences of the plant genome, self-sustained sequence replication, simple sequence repeat (SSR), single nucleotide polymorphism (SNP), or amplified fragment length polymorphisms (AFLP).

The majority of genetic markers rely on one or more properties of nucleic acids for their detection. For example, some techniques for detecting genetic markers utilize hybridization of a probe nucleic acid to nucleic acids corresponding to the genetic marker. Hybridization formats include, for example, but are not limited to, solution phase, solid phase, mixed phase, or in situ hybridization assays. Markers which are restriction fragment length polymorphisms (RFLP), are detected by hybridizing a probe which is typically a sub-fragment (or a synthetic oligonucleotide corresponding to a sub-fragment) of the nucleic acid to be detected to restriction digested genomic DNA. The restriction enzyme is selected to provide restriction fragments of at least two alternative (or polymorphic) lengths in different individuals, and will often vary from line to line. Determining a (one or more) restriction enzyme that produces informative fragments for each cross is a simple procedure known in the art. After separation by length in an appropriate matrix (e.g., agarose) and transfer to a membrane (e.g., nitrocellulose, nylon), the labeled probe is hybridized under conditions which result in equilibrium binding of the probe to the target followed by removal of excess probe by washing.

Nucleic acid probes to the marker loci can he cloned and/or synthesized. Detectable labels suitable for use with nucleic acid probes include, for example, but are not limited to, any composition detectable by spectroscopic, radio-isotopic, photochemical, biochemical, immunochemical, electrical. optical or chemical means. Useful labels include, for example, biotin for staining with labeled streptavidin conjugate, magnetic beads, fluorescent dyes, radiolabels, enzymes, and colorimetric labels. Other labels include ligands which bind to antibodies labeled with fluorophores, chemiluminescent agents, and enzymes. Labeling markers is readily achieved such as, for example, by the use of labeled PCR primers to marker loci.

The hybridized probe is then detected using any suitable technique known in the art, for example autoradiography or other similar detection technique (e.g., fluorography, liquid scintillation counter). Examples of specific hybridization protocols are known in the art.

Amplified variable sequences may refer to amplified sequences of the plant genome which exhibit high nucleic acid residue variability between members of the same species. Organisms have variable genomic sequences and each organism has a different set of variable sequences, Once identified, the presence of specific variable sequences can be used to predict phenotypic traits. Preferably, DNA from the plant serves as a template for amplification with primers that flank a variable sequence of DNA. The variable sequence is amplified and then sequenced.

In vitro amplification techniques are known in the art. Examples of such techniques include, but are not limited to, the polymerase chain reaction (PCR), the ligase chain reaction (LCR), Qβ-replicase amplification and other RNA polymerase mediated techniques (e.g., NASBA), and are found in Sambrook et al, 2001, Molecular Cloning, A Laboratory Manual, Codd Spring Harbor Press, N.Y. and Ausubel et al., 1989, Current Protocols in Molecular Biology, Green Publishing Associates and Wiley .interscience, N.Y., as well as .Mullis el al. (1987) U.S. Pat. No, 4,683,202; PCR Protocols, A Guide to Methods and Applications (Innis et al., eds.) Academic Press Inc., Sari Diego Academic Press Inc, San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. A.cad, Sci. USA 86, 1173; Guatelli et al. (1990) Proc. Natl. Acad. Sci. USA 87, 1874; Lomell el al. (1989) J. Clin. Chem. 35, 1826; Landegren el at., (1988) Science 241, 1077-1080; Van Brunt (1990) Biotechnology 8, 291-294; Wu and Wallace, (1989) Gene 4, 560; Barringer et at. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684, and the references therein, in which PCR amplicons of up to 40 kb are generated.

Oligonucleotides for use as primers, e.g., in amplification reactions and for use as nucleic acid sequence probes are typically synthesized chemically according to, for example, the solid phase phosphoramidite triester method described by Beaucage and Caruthers (1981) Tetrahedron Lett. 22:1859.

Alternatively, self-sustained sequence replication can be used to identify genetic markers. Self-sustained sequence replication refers to a method of nucleic acid amplification using target nucleic acid sequences which are replicated exponentially in vitro under substantially isothermal conditions by using three enzymatic activities involved in retroviral replication: (1) reverse transcriptase, (2) RNAase H, and (3) a DNA-dependent RNA polymerase (Guatelli et al. (1990) Proc Natl Acad Sci USA 87:1874). By mimicking the retroviral strategy of RNA replication by means of cDNA intermediates, this reaction accumulates cDNA and RNA copies of the original target.

Amplified fragment length polymorphisms (AFLP) can also be used as genetic markers (as described in Vos et al. (1995) Nucl Acids Res 23:4407). The phrase “amplified fragment length polymorphism” refers to selected restriction fragments which are amplified before or after cleavage by a restriction endonuclease. The amplification step allows easier detection of specific restriction fragments. AFLP allows the detection of large numbers of polymorphic markers and has been used for genetic mapping of plants (as described in Becker et at. (1995) Mol Gen Genet. 249:65; and Meksem et at. (1995) Mol Gen Genet. 249:74),

Allele-specific hybridization (ASH) can he used to identify the genetic markers of the invention, ASH technology is based on the stable annealing of a short, single-stranded, oligonucleotide probe to a completely complementary single-strand target nucleic acid. Detection is via an isotopic or non-isotopic label attached to the probe.

For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization. In this manner, only one of the alternative probes will hybridize to a target sample that is homozygous or homogenous for an allele. Samples that are heterozygous or heterogeneous for two alleles will hybridize to both of two alternative probes.

ASH markers are used as dominant markers where the presence or absence of only one allele is determined from hybridization or lack of hybridization by only one probe. The alternative allele may be inferred from the lack of hybridization. ASH probe and target molecules are optionally RNA or DNA; the target molecules are any length of nucleotides beyond the sequence that is complementary to the probe; the probe is designed to hybridize with either strand of a DNA target; the probe ranges in size to conform to variously stringent hybridization conditions, etc.

PCR allows the target sequence for ASH to be amplified from low concentrations of nucleic acid in relatively small volumes. Otherwise, the target sequence from genomic DNA is digested with a restriction endonuclease and size separated by gel electrophoresis. Hybridizations typically occur with the target sequence bound to the surface of a membrane or, as described, for example, in U.S. Pat. No, 5,468,613, the ASH probe sequence may be bound to a membrane.

In one embodiment, ASH data are obtained by amplifying nucleic acid fragments (amplicons) from genomic DNA using PCR, transferring the amplicon target DNA to a membrane in a dot-blot format, hybridizing a labeled oligonucleotide probe to the amplicon target, and observing the hybridization dots by autoradiography.

Single nucleotide polymorphisms (SNP) are markers that consist of a shared sequence differentiated on the basis of a single nucleotide. Typically, this distinction is detected by differential migration patterns of an amplicon comprising the SNP on e.g., an acrylamide gel. However, alternative modes of detection, such as hybridization, e.g., ASH, or RFLP analysis, are not excluded.

In yet another basis for providing a genetic linkage map, Simple sequence repeats (SSR), take advantage of high levels of di-, tri-, or tetra-nucleotide tandem repeats within a genome. Dinucleotide repeats have been reported to occur in the human genome as many as 50,000 times with n varying from 10 to 60 or more (as described in Jacob et al. (1991) Cell 67:213). Dinucleotide repeats have also been found in higher plants (as described in Condit and Hubbell (1991) Genome 34:66).

Briefly, SSR data is generated by hybridizing primers to conserved regions of the plant genome which flank the SSR sequence. PCR is then used to amplify the dinucleotide repeats between the primers. The amplified sequences are then electrophoresed to determine the size and therefore the number of di-, tri- and tetra-nucleotide repeats.

Alternatively, isozyme markers are employed as genetic markers. Isozymes are multiple forms of enzymes which differ from one another in their amino acid, and therefore their nucleic acid sequences. Some isozymes are multimeric enzymes containing slightly different subunits. Other isozyrnes are either multimeric or monomeric but have been cleaved from the proenzyme at different sites in the amino acid sequence. Isozymes can be characterized and analyzed at the protein level, or alternatively, isozymes which differ at the nucleic acid level can be determined. In such cases any of the nucleic acid based methods described herein can be used to analyze isozyme markers.

In alternative embodiments, in silico methods can be used to detect the marker loci. For example, the sequence of a nucleic acid comprising the marker can be stored in a computer. The desired marker locus sequence or its homolog can be identified using an appropriate nucleic acid search algorithm as provided by, for example, in programs as BLAST or any suitable sequence alignment tool.

### Sesame Plants

The sesame plants described herein are not naturally occurring sesame plants. Breeding efforts during the last seventy years have attempt to breed a mechanically harvestable sesame plant capsule using single gene mutations (ID, GS) and even a combination of a few genes (ND and IND varieties). These efforts have failed, with the majority of the world's sesame (over 99%) being dehiscent(shattering) type. One reason is that the breeding varieties were developed using classical breeding methodology. Despite the breeding efforts done by other breeders/breeding companies, the sesame plant varieties derived from those breeding programs still have poor performance and many agronomical problems such as low germination, plant lodging and low yield potential.

The present invention also provides a shattering-resistant sesame plant selected by screening for shattering-resistance capsules plant, the selection comprising interrogating genomic nucleic acids for the presence of a marker molecule that is genetically linked to an allele of a QTL, associated with shattering-resistance capsules in the sesame plant, where the allele of a QTL is also located on a linkage group associated with shattering-resistant sesame. A sesame plant or part thereof may comprise at least one quantitative trait loci (“QTLs”) associated with shattering-resistant capsules, wherein the QTLs comprises QTLs 1 to 8. The sesame plant or part thereof may comprise at least three quantitative trait loci (“QTLs”) associated with shattering-resistant capsules, wherein the QTLs comprises QTLs 1 to 8. The sesame plant or part thereof may comprise at least one, two, three, four, five, six, seven or eight quantitative trait loci (“QTLs”) associated with shattering-resistant capsules, wherein the QTLs comprises QTLs 1 to 8.

Shattering-resistant capsules are measured in fully developed capsules having at most 10% seed moisture, and the shatter-resistant capsules are characterized by at least one of the following features: (i) at least 80% seed retention after shaking the plant, (ii) at least 80% seed retention after the capsules are turned upside down, (iii) a ratio of at least 5:1 between a total length of the capsule and a length of a zone in which the capsule tips are open, and/or 20-30% of the capsules retain 90-95% of the seeds in developed green capsules before drying.

Plants of the invention can be part of or generated from a breeding program. The choice of breeding method may depend on the mode of plant reproduction, the heritability of the trait(s) being improved, and the type of cultivar used commercially (e.g., F1 hybrid cultivar, pureline cultivar). A cultivar may refer to a race or variety of a plant that has been created or selected, and maintained through cultivation.

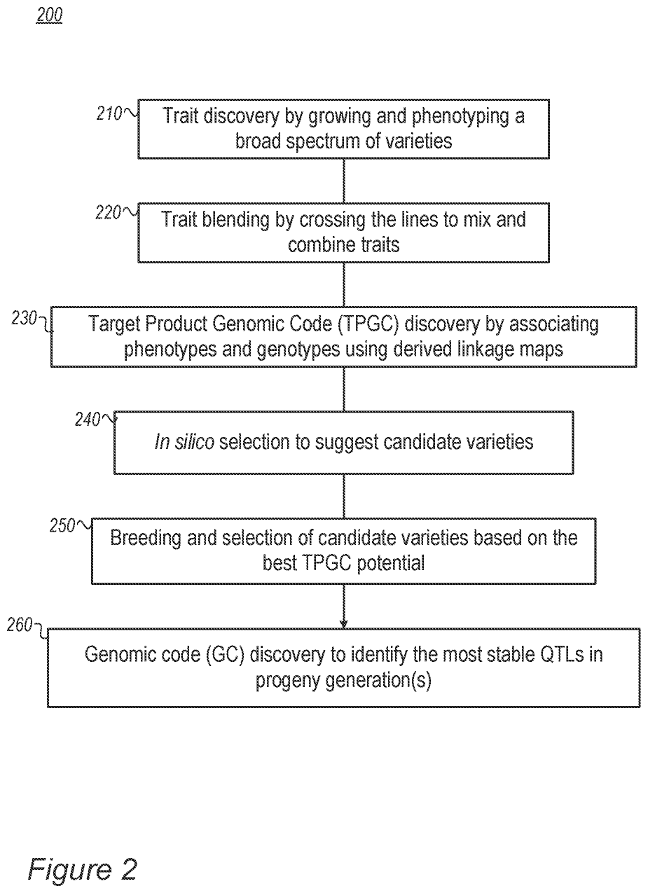

FIG. 2 is a high-level schematic illustration of a computationally supported breeding method 200, according to some embodiments of the invention. Method 200 may be at least partially implemented by at least one computer processor; computationally supported breeding method 200 is used to detect and combine QTLs from a plurality of sesame varieties to develop disclosed sesame plants with shatter-resistant capsules , hich are different than any of the parent varieties by virtue of the achieved phenotypical and/or yield characteristics.

Computationally supported breeding method 200 comprises stages of trait discovery by growing and phenotyping a broad spectrum of varieties (stage 210), trait blending by developing hybridized lines through crossing the selected lines to mix and combine traits and selling of the progeny in subsequent generations (stage 220), Target Product Genomic Code (TPGC) discovery by associating phenotypes and genotypes using derived linkage maps (stage 230), in silico selection to suggest candidate varieties (stage 240), breeding candidate varieties and selection of varieties based on the best TPGC potential (stage 250) and genomic code (GC) discovery to identify the most stable QTLs in hybridized progeny generation(s) (stage 260), as explained in detail below. TPGC discovery 230, in silico validation 250 and GC discovery 260 are based on computational algorithms that cannot be performed manually and provide the computational support for the judicious selection of the varieties that are generated and further crossed during the development process to yield disclosed sesame plants with shatter-resistant capsules. It is noted that during the discovery phase, the QTLs are derived in order to combine them (by hybridization) is create unique combinations of QTLs which do not exist in known world lines. The unique combinations of QTLs (cassettes) have an additive effect on the measured traits (e.g., shattering-resistant traits) and produce the disclosed sesame plants having shatter-resistant capsules according to the present invention.

In certain embodiments, sesame lines were bred to reach shattering-resistance by collecting various sesame lines worldwide, creating F2 linkage populations, applying intensive phenotypinrg and genotyping of thousands of sesame lines, predicting of QTLs affecting the shattering-resistance trait, and establishing unique marker combinations, termed “marker cassettes” herein, to characterize novel shattering-resistant lines found by the methods described herein and not existing in commercial or natural lines.

The breeding methodology was based on algorithms for deriving the Target Product Genomic Code (TPGC) to associate (i) the Target Product (TP) being defined in advance based on market requirements and including a set of desired attributes (traits) that are available in natural genetic variations; and (ii) the Genomic Code (GC) comprising set(s) of genomic regions that include quantitative trait loci (QTLs) that affect and are linked to the TP traits. The algorithms may be configured to calculate multiple genomic interactions and to maximize the genomic potential of specific plants for the development of new varieties. The breeding program was constructed to derive the TPGC, and then by crossing and selling to achieve a product which contains the specific GC that corresponds to the required TPs.

Certain embodiments of the breeding process of developing lines, through crossing and successive generations of selling comprise stages such as: (i) Trait Discovery, in which a broad spectrum of varieties from different geographies and worldwide sources are grown and phenotyped in order to discover new traits that can potentially be combined to create new varieties; (ii) Trait Blend, in which a crossing cycle is carried out based on phenotypic assumption(s), in which the different traits are mixed and combined. Initial trait cycle(s) are followed by additional cycle(s) to create F2 (and possibly higher generations) population(s) that provide the basis for algorithmic analysis for constructing the TPGC; (iii) TPGC Discovery, in which the plant(s) are phenotyped and genotyped to produce linkage map(s), discovering the relevant QTLs and deriving the TPGC; (iv) several line validation stages over several years in which sesame lines based on millions of in silky) calculated variations (and/or selections) grown and are used to define the initial varieties; (v) Trait TPGC Blend, in which accurate crossings are performed in order to calculate the most efficient way to reach the best TPGC. The crossings are performed after in silico selection from millions of combinations, and are based, at least on part on phenotype assumptions; and (vi) Consecutive algorithm-based GC discovery stage(s) applied to F2 (or higher generation) population(s) grown in additional cycle(s).

Defining the TP for shatter-resistant sesame varieties includes the development of high throughput methods for identification of shattering-resistance.

In the following non-limiting example of the process, Trait Discovery (i) was based on proprietary germplasm including hundreds of elite varieties and thousands of F2 individual plants and also 120 different sesame lines that were obtained from the U.S. National Plant Germplasm. System (NPGC) and courtesy of professor Arnram Ashri's sesame germplasm collection (see Ashri, A. 1998, Sesame Breeding. In: Janick J. (ed.), Plant Breeding Reviews Vol. 16. John Wiley and Sons, Somerset, N.J., pp. 179-228). These lines were used for the Trait Blend stage (ii), with crosses executed based on the potential for enrichment of genomic diversity to create new complex(es) of traits for the shattering-resistance as the initial step for the TP-directed breeding program for shattering-resistant sesame lines. The resulting F1 hybrids were :later self-crossed to create F2 linkage populations that showed phenotypic segregation. The F2 population were then planted in three different environments for discovering the TPGC (iii) that includes shattering-resistance traits. After screening and deep phenotyping of 5000 individuals, a set of ca. 400 shattering resistant representatives was selected. The selected individuals from the F2 population were further massively phenotyped for traits associated with shattering-resistance, as detailed in the following. The measurement results were summarized into the representative shattering-resistance trait.

TPGC Discovery (iii) included genotyping ca. 5000 selected individual plants from ten populations. The analysis was performed with a panel of ca. 500 markers based on single nucleotide polymorphism (SNP) and directly designed based on the polymorphism found in the parental lines of the populations which were analyzed in depth using high throughput DNA sequencing technologies. The panel was designed to maximize the chance to have the largest number of common segregating SNPs in order to create highly similar linkage maps for gill observed populations. The computation of linkage maps was executed on each linkage F2 population based on the genotyping results. Linkage maps were computed with MultiPoint, an interactive package for ordering multilocus genetic maps, and verification of maps based on resampling techniques. Discovery of QTLs that are related to shattering-resistance was carried out with the MultiQTL package, based on the linkage maps that were merged by Multipoint and the F2 population phenotype data, and using multiple interval mapping (MIM). The significance and co-occurrences of the shattering-resistance markers were evaluated using an algorithm that related the genotype-phase of each marker to respective QTLs and traits in linkage maps of the 10 F2 populations (also called “linkage F2 populations” herein) in each population, for populations in different environments. QTL significance was computed with permutation, bootstrap tools and FDR (false discovery rate) for total analysis. The linkage maps of all ten F2 populations and the information of the shattering-resistance traits over all genotyped plants belonging to those populations were analyzed and used to predict the QTLs in a “one trait to one marker” model, in which for all markers that constructed the linkage maps, each trait was tested independently against each one of the markers. In the provided examples, altogether eight markers were found to be related to traits associated shattering-resistance.

In general, the ten linkage F2 populations presented different markers that related to shattering-resistance. However, subsets of common markers were found to be shared by multiple populations and are referred to herein as marker cassettes.

It is emphasized that the breeding process is explained using non-limiting examples from a specific part of the breeding program, and is not limited to the specific populations and varieties derived by this specific part of the breeding program. For example, different F2 population may be bred and used to derive additional varieties that are characterized by one or more of the disclosed QTLs.

Following TPGC Discovery (iii), an in-silica breeding program (iv) was established to process the TPGC blend (including combinations of QTLs for different plants) to simulate and predict the genotypic states of self, cross-self and hybrid plant with respect to the QTLs and their predicted effects on each phase of the markers for the shattering-resistance trait. The in-silica breeding program was constructed to yield millions of in silica selfing combinations which were bred and evaluated in-silica up to F8 to measure the potential for each of the genotyped plants to acquire the shattering-resistance traits in the right combination at the right phase. The analysis resulted in identifying ca. 400 F2 plants having the highest score for shattering-resistance, which were thus chosen for the actual selling and cross-selfing procedures. The F3 seeds from these selected F2 plants were sown in plots in the subsequent growing season. Under this procedure, QTLs from different populations were combined to yield F3 plants containing new and unique cassettes of QTLs and resulting in shattering-resistance.

The shattering-resistant sesame lines were then validated as retaining the traitin the following generations by genotyping the F3 and some subsequent generation offspring to verify they maintained the identified marker cassettes. Specifically, the parental lines of linkage F2 populations together with 120 different sesame cultivars (landraces and old commercial varieties) were genotyped based on shattering-resistance markers of all populations.

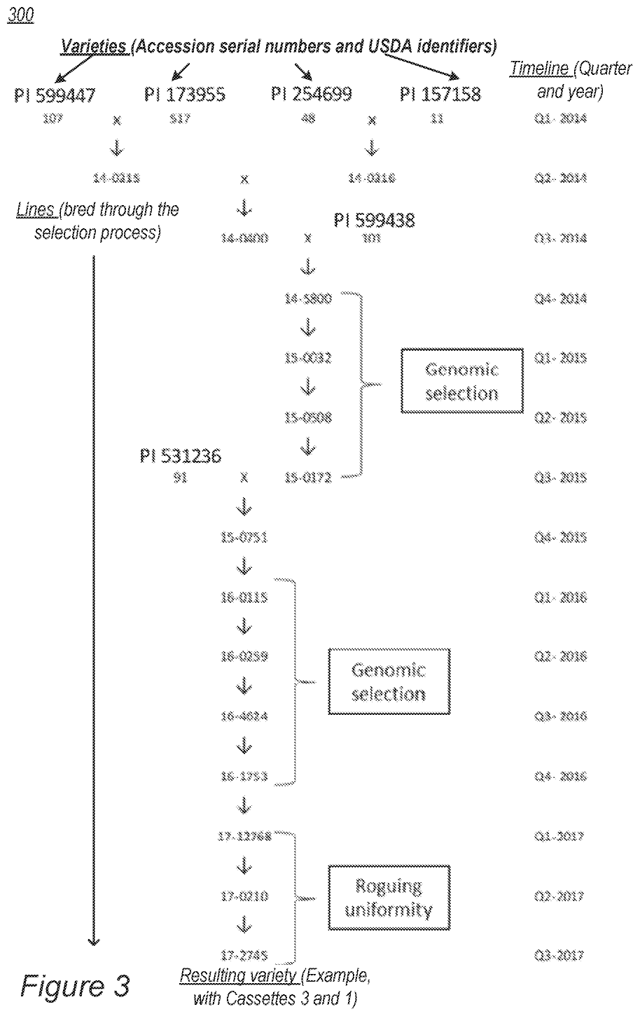

FIG. 3 provides a non-limiting example of a genealogy 300 of one of the disclosed sesame varieties, illustrating schematically the complex breeding process outlined herein, according to some embodiments of the invention. As illustrated in FIG. 3, multiple crosses were judiciously performed to reach specific QTL, combinations of cassettes 3 and 1, followed by multiple generations for genetic stabilization and additional crossing and stabilization. The non-limiting genealogy illustrates schematically the development of registered variety ES108, from a plurality of varieties (identified by their accession serial numbers and. USDA identifiers) which were judiciously bred over a period of three and a half years to yield multiple generations in which genomic selection and stabilization were applied.

The inventors note that none of the sesame plants with shatter-resistant capsules that were bred according to the methods described herein is naturally occurring; indeed, they were derived by highly complicated computationally-supported breeding methods 200 described above, in which the genotypes of multiple sesame varieties were judiciously combined and analyzed, to discover and accumulate the recited QTL, markers and corresponding phenotypic traits. Although the recited sesame plants are not genetically modified by sequences originating from other species, they cannot be reached merely by natural processes, as is evident by the detailed and intentional breeding program that was applied to specifically measure required characteristics, detect corresponding markers using bioinformatics methods and combine the detected QTLs in the selected varieties by classic breeding approaches. The inventors note that due to the huge complexity of the breeding program, involving growing, selecting and breeding of hundreds of varieties over many generations in the field, and based on genetic analysis of the varieties and of the relations of markers to phenotypic characteristics, this breeding process cannot happen merely by natural means and therefore cannot be considered a natural phenomenon. It is further noted that due to the shatter-resistance characteristics, disclosed varieties are severely hindered from natural plant propagation. Finally, it is noted that while the disclosed QTL, markers are not heterologous to sesame as a species, the identified QTLs are not present in the recited combinations in any of over 250 prior art varieties which were used as initial breeding stock (i.e., the QTL combinations in the disclosed sesame varieties do not exist in sesame world germplasm), and that the QTLs genomics of the sesame plants has been significantly and judiciously modified by the breeding program. Therefore, at the taxonomic level of the varieties, the shattering-resistant sesame plants may be considered hybridized in that the QTL markers are mixed and introduced from other sesame varieties. The offspring of the herein disclosed varieties will show the same QTL composition (as long as the QTL is homozygous) and the same shattering resistance.

In certain embodiments, methods for producing a sesame plant or seed, or a group of plants or seeds, are provided, whereby the plant, or group of plants, produce(s) a seed may comprise one or more introgressed shattering-resistant capsule loci associated with a plurality of quantitative trait loci (“QTLs”) associated with shattering-resistant capsules, wherein said plurality of QTLs comprises QTLs 1 to 8. The method comprises crossing two parent sesame plants or selfing a sesame plant and harvesting the resulting sesame seeds, from the cross or selfing, wherein at least one parent is a sesame plant as described herein, or a derivative thereof, Seeds produced by the method are also provided herein, as are sesame plants produced by growing those seeds and sesame capsules harvested from those grown plants.

The methods may further comprise the step of growing an F1 hybrid sesame plant obtained from seed obtained from said cross, crossing the F1 sesame plant to another sesame plant, e.g., to one of the parents used, and selecting progeny sesame plants comprising one or more introgressed shattering-resistant capsule loci associated with a plurality of quantitative trait loci (“QTLs”) associated with shattering-resistant capsules, wherein said plurality of QTLs comprises QTLs 1 to 8.

The methods may comprise the steps of:
(a) crossing a sesame plant producing sesame seeds comprising one or more introgressed shattering-resistant capsule loci associated with a plurality of quantitative trait loci (“QTLs”) associated with shattering-resistant capsules, wherein said plurality of QTLs comprises QTLs 1 to 8,
(b) obtaining the F1 seeds from said cross,
(c) selfing and/or crossing the plants obtained from F1 seeds one or more times with one another or with other sesame plants,
(d) identifying and selecting progeny plants which produce seeds comprising one or more introgressed shattering-resistant capsule loci associated with a plurality of quantitative trait loci (“QTLs”) associated with shattering-resistant capsules, wherein said plurality of QTLs comprises QTLs 1 to 8; and
(e) phenotyping the seeds.

Optionally, steps (c) and/or (d) can be repeated several times. Crossing in step (c) may also involve backcrossing. In step (d), plants comprising one or more introgressed shattering-resistant capsule loci associated with a plurality of quantitative trait loci (“QTLs”) associated with shattering-resistant capsules, wherein said plurality of QTLs comprises QTLs 1 to 8 may be selected. Thus, the one or more introgressed shattering-resistant capsule loci associated with a plurality of quantitative trait loci (“QTLs”) associated with shattering-resistant capsules, wherein said plurality of QTLs comprises QTLs 1 to 8 can also be used as selection criteria in addition to or as an alternative of shattering-resistant capsule traits. The same applies to the methods described herein below, even if only shattering-resistant traits are measured.

Phenotyping may comprise detecting one or more introgressed shattering-resistant capsule loci associated with a plurality of quantitative trait loci (“QTLs”) associated with shattering-resistant capsules, wherein said plurality of QTLs comprises QTLs 1 to 8, in the seeds (e.g., by phenotyping one or more populations of step c) above) and selecting rare recombinants or mutants which comprise one or more introgressed shattering-resistant capsule loci associated with a plurality of quantitative trait loci (“QTLs”) associated with shattering-resistant capsules, wherein said plurality of QTLs comprises QTLs 1 to 8. The plants used under a) may be commercially available sesame plant cultivars or breeding lines. Phenotyping can be carried out on a plurality of single seeds independently, preferably grown under the same conditions next to suitable controls, or on a sample composed of (all or parts of) several seeds. When a single seed is used, preferably the mean value is calculated from a representative number of seeds. Phenotyping can be done one or more times. Phenotyping can be carried out at one or more steps of a breeding scheme.

Phenotyping may also comprise an analysis of the one or more introgressed. shattering-resistant capsule loci associated with a plurality of quantitative trait loci (“QTLs”) associated with shattering-resistant capsules, wherein said plurality of QTLs comprises QTLs 1 to 8 in the sesame plants produced.

A method for making sesame plants comprising one or more introgressed shattering-resistant capsule loci associated with a plurality of quantitative trait loci (“QTLs”) associated with shattering-resistant capsules, wherein said plurality of QTLs comprises QTLs 1 to 8 may comprise:
(a) optionally, analyzing sesame seeds and/or capsules for one or more introgressed shattering-resistant capsule loci associated with a plurality of quantitative trait loci (“QTLs”) associated with shattering-resistant capsules, wherein said plurality of QTLs comprises QTLs 1 to 8,
(b) crossing plants producing seeds comprising one or more introgressed. shattering-resistant capsule loci associated with a plurality of quantitative trait loci (“QTLs”) associated with shattering-resistant capsules, wherein said plurality of QTLs comprises QTLs 1 to 8 with sesame plants to produce F1 hybrids,
(c) selfing and/or (back)crossing F1 hybrid plants one or more times,
(d) selecting progeny plants comprising one or more introgressed shattering-resistant capsule loci associated with a plurality of quantitative trait loci (“QTLs”) associated with shattering-resistant capsules, wherein said plurality of QTLs comprises QTLs 1 to 8 (at harvest and/or after storage), and also having shattering-resistant capsules; and
(e) selecting a sesame plant producing seeds comprising one or more introgressed shattering-resistant capsule loci associated with a plurality of quantitative trait loci (“QTLs”) associated with shattering-resistant capsules, wherein said plurality of QTLs comprises QTLs 1 to 8, Step d) may involve genetic analysis at harvest and/or after storage. in the initial cross, the sesame parent may be a sesame variety, cultivar or breeding line and the other plant may be a sesame variety, cultivar or breeding line. Preferably steps (c) and (d) are repeated several times, so that several cycles of phenotypic recurrent selection are carried out, leading to sesame plants of step (e).

A method of producing an inbred sesame plant comprising one or more introgressed shattering-resistant capsule loci associated with a plurality of quantitative trait loci (“QTLs”) associated with shattering-resistant capsules, wherein said plurality of QTLs comprises QTLs 1 to 8, the method may comprise:
(a) the creation of variable populations of Sesamum indican comprising the steps of crossing a Sesamum indicum plant or plants producing seeds comprising one or more introgressed shattering-resistant capsule loci associated with a plurality of quantitative trait loci (“QTLs”) associated with shattering-resistant capsules, wherein said plurality of QTLs comprises QTLs 1 to 8 with a plant of the species Sesamum indicum,
(b) harvesting the F1 seed from any of the plants used in the cross of (a) and growing F1 planus from the seed harvested.
(c) selfing the plants grown under b) or crossing these plants amongst one another, or crossing these plants with the plant or plants of Sesamum indicum,
(d) growing plants from the resulting seed harvested under noimal plant growing conditions and,
(e) selecting plants producing seeds comprising one or more introgressed shattering-resistant capsule loci associated with a plurality of quantitative trait loci (“QTLs”) associated with shattering-resistant capsules, wherein said plurality of QTLs comprises QTLs 1 to 8, followed by selfing the selected plants, and optionally
(f) repeating the steps (d) and/or (e) until the inbred lines are obtained which are homozygous and can be used as parents in the production of sesame plant hybrids comprising one or more introgressed shattering-resistant capsule loci associated with a plurality of quantitative trait loci (“QTLs”) associated with shattering -resistant capsules, wherein said plurality of QTLs comprises QTLs 1 to 8.

A method for producing a sesame seed crop from sesame seeds or plants according to the invention and sesame seeds harvested therefrom is provided.

Containers may comprise a plurality of sesame seeds and/or sesame capsules having the phenotypes described herein, as well as containers comprising a plurality of sesame seeds of the above plants or containers comprising a plurality of sesame plants or seedlings. Containers may be of any type, such as bags, cans, tins, trays, boxes, flats. A container may contain at least about 1 pound, 5 pounds, 10 pounds or more of sesames seeds. The container may be stored in any location, e.g., a store (a grocery store), warehouse, market place, food processor, or distributor.

A sesame plant or a part thereof may comprise at least one introgressed shattering-resistant capsule loci associated with a plurality of quantitative trait loci (“QLs”) associated with shattering-resistant capsules, wherein said plurality of QTLs comprises QTLs 1 to 8. The sesame plant or part thereof may comprise at least three introgressed shattering-resistant capsule loci associated with a plurality of quantitative trait loci (“QTLs”) associated with shattering-resistant capsules, wherein the QTLs comprise QTLs 1 to 8.

The sesame plant may have shattering-resistant capsules which are full or partial shattering-resistant capsules.

The sesame plant or a part thereof comprises an allele of each of a plurality of markers associated with said QTLs.

The “part thereof” may be a seed, an endospettn, an ovule, pollen, cell, cell culture, tissue culture, plant organ, protoplast, meristem, embryo, or a combination thereof.

The sesame plant or part may be a hybrid.

Certain embodiments comprise a seed of the sesame plant comprising one or rlore introgressed shattering-resistant capsule loci associated with a plurality of quantitative trait loci (“QTLs”) associated with shattering-resistant capsules, wherein said plurality of QTLs comprises QTLs 1 to 8.

Certain embodiments comprise a cell of the sesame plant comprising: one or more introgressec shattering-resistant capsule loci associated with a plurality of quantitative trait loci (“QTLs”) associated with shattering -resistant capsules, wherein said plurality of QTLs comprises QTLs 1 to 8.

Certain embodiments comprise a cell of the sesame plant comprising one or more introgressed shattering-resistant capsule loci associated with a plurality of quantitative trait loci (“QTLs”) associated with shattering-resistant capsules, wherein said plurality of QTLs comprises QTLs 1 to 8.

Certain embodiments comprise a sesame plant grown frorn a seed comprising one or more introgressed shattering-resistant capsule loci associated with a plurality of quantitative trait loci (“QTLs”) associated with shattering-resistant capsules, wherein said plurality of QTLs comprises QTLs 1 to 8.

Certain embodiments comprise a method for producing a hybrid sesame seed plant comprising crossing the sesame plant comprising one or more introgressed shattering-resistant capsule loci associated with a plurality of quantitative trait loci (“QTLs”) associated with shattering-resistant capsules, wherein said plurality of QTLs comprises QTLs 1 to 8 with another sesame plant, and obtaining an F1 sesame plant, wherein the F1 sesame plant comprises one or more introgressed shattering-resistant capsule loci associated with a plurality of quantitative trait loci (“QTLs”) associated with shattering-resistant capsules, and wherein said plurality of QTLs comprises QTLs 1 to 8.

Certain embodiments comprise sesame plants grown from the F1 sesame plant, wherein the F1 sesame plant comprises one or more introgressed shattering-resistant capsule loci associated with a plurality of quantitative trait loci (“QTLs”) associated with shattering-resistant capsules, and wherein said plurality of QTLs comprises QTLs 1 to 8.

Certain embodiments comprise a method for producing sesame plants or seeds comprising growing a sesame plant from the F1 seeds, crossing the F1 sesame plant with a sesame plant, and obtaining F2 seeds from the cross.

Certain embodiments comprise a capsule of the sesame plant which may comprise one or more introgressed shattering-resistant capsule loci associated with a plurality of quantitative trait loci (“QTLs”) associated with shattering-resistant capsules, wherein said plurality of QTLs comprises QTLs 1 to 8.

Certain embodiments comprise a method of producing sesame seeds may comprise planting seeds for a sesame plant comprising one or more introgressed shattering-resistant capsule loci associated with a plurality of quantitative trait loci (“QTLs”) associated with shattering-resistant capsules, wherein said plurality of QTLs comprises QTLs 1 to 8, growing, and harvesting the sesame seeds or capsules. The harvesting may be done by machine.

Certain embodiments comprise a field comprising the sesame plant which may comprise one or more introgressed shattering-resistant capsule loci associated with a plurality of quantitative trait loci (“QTLs”) associated with shattering-resistant capsules, wherein said plurality of QTLs comprises QTLs 1 to 8.

Sesame seeds and other plant parts described herein can be further processed by a method known to one of skilled in the art. This method may comprise heat treating, for example roasting, the pliant parts, preferably sesame seeds. The method may further comprise comminuting, e.g., grinding, the seeds, including seeds following heat treatment (roasting).

Plant breeding methods are described in the art and fully described, for example, in U.S. Pat. Nos. 8,779,233; 6,670,524; 8,692,064; 9,000,258; 8,987,549; 8,637,729; 6,455,758; 5,981,832; 5,492,547; 9,167,795; 8,656,692; 8,664,472; 8,993,835; 9,125,372; 9,144,220; 9,462,820; and U.S. Patent Application Publication Nos 2015/0082476; 2011/0154528; 2014/0215657; 2017/0055481; 2015/0150155; and 2015/0101073, each of which is incorporated by reference herein in its entirety.

Approaches for breeding the plants are described in the art, Selected, non-limiting approaches for breeding the plants are described below. A breeding program can he enhanced using marker assisted selection (MAS) of the progeny of any cross. It is further understood that any commercial and non-commercial cultivars can be utilized in a breeding program.

For highly inheritable traits, a choice of superior individual plants evaluated at a single location can be effective, whereas for traits with low heritability, selection can be based on mean values obtained from replicated evaluations of families of related plants. Popular selection methods commonly include, for example, but are not limited to, pedigree selection, modified pedigree selection, mass selection, and recurrent selection. In a preferred embodiment, a backcross or recurrent breeding methods can be used.

The complexity of inheritance influences choice of the breeding method. Backcross breeding can be used to transfer one or a few favorable genes for a highly heritable trait into a desirable cultivar. This approach has been used extensively in breeding. Various recurrent selection techniques are used to improve quantitatively inherited traits controlled by numerous genes. The use of recurrent selection in self-pollinating crops depends on the ease of pollination, the frequency of successful hybrids from each pollination event, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its perform nance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new sesame cultivars requires the development and selection of sesame varieties, the crossing of these varieties and selection of superior hybrid crosses. The hybrid seed can be produced by manual crosses between selected male-fertile parents or by using male sterility systems, or by using differences between maternal and parental traits heritability in the seed as described in Israel Patent Application Publication IL239702, which is incorporated by reference herein in its entirety. Hybrids are selected for certain single gene traits such as, for example, herbicide resistance which indicate that the seed is truly a hybrid. Additional data on parental lines, as well as the phenotype of the hybrid, may influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selling and selection of desired phenotypes. New cultivars can be evaluated to determine which have commercial potential.

Pedigree breeding is used commonly for the improvement of self-pollinating crops. Two parents who possess favorable, complementary traits are crossed to produce an F1. Ans F2 population is produced by selfing one or several F1's. Selection of the best individuals in the best families is selected. Replicated testing of families can begin in the F4 generation to improve the effectiveness of selection for traits with low heritability. At an advanced stage of inbreeding (e.g., F6 and F7), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant, is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

Other suitable methods such as, for example, single-seed descent procedure and a multiple-seed procedure can also be used.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines in each generation due to failure of some seeds to germinate or some plants to produce at least one seed. As a result, not all of the F2. plants originally sampled in the population will be represented by a progeny when generation advance is completed.

In a multiple-seed procedure, breeders commonly harvest one or more capsules from each plant in a population and thresh them together to form a bulk. Part of the bulk is used to plant the next generation and part is put in reserve.

Other breeding methods are described in the art, for example, in Fehr, Pzinciples of Cultivar Development Vol. 1, (1987), which is incorporated by reference herein in its entirety.

The present invention also provides for parts of the plants of the present invention. Plant pails without limitation, include seed. endosperm, ovule and pollen. In a particular embodiment of the present invention, the plant part is a seed.

Plants or parts thereof of the present invention may be grown in culture and regenerated. Methods for the regeneration of sesame plants from various tissue types and methods for the tissue culture of sesame are known in the art (See, for example, George et al., 1987, Ann Bot., vol. 60 (1), pages 17-21; Were et al., 2006, Plant Cell Tiss Organ Cult, vol. 85, page 235, each of which is incorporated by reference herein in its entirety). The present invention also provides a shattering-resistant sesame plant selected for by screening for a plant having shattering-resistant capsules, the selection comprising interrogating genomic nucleic acids for the presence of a marker molecule that is genetically linked to an allele of a QTL associated with shattering-resistant capsules in the sesame plant, where the allele of a QTL is also located on a linkage group associated with shattering-resistant sesame.

The genetic linkage of additional marker molecules can be established by a gene mapping model such as, for example, without limitation, the flanking marker model reported by Lander and Botstein, Genetics, 121:185-199 (1989), and the interval mapping, based on maximum likelihood methods described by Lander and 13otstein, Genetics, 121:185-199 (1989), and implemented in the software package MAPMAKER/QTL (Lincoln and Lander), Mapping Genes Controlling Quantitative Traits Using MAPMAKER/QTL, Whitehead .institute for Biomedical Research, Massachusetts, (1990), each of which is incorporated by reference herein in its entirety. Additional software includes Qgene, Version 2.23 (1996), Department of Plant Breeding and Biometry, 266 Emerson Hall, Cornell University, Ithaca, N.Y., the manual of which is incorporated by reference herein in its entirety. Additional software includes MultiPoint program (Yefim Ronin, Natural Science, Vol. 2, No. 6, 576-589 (2010), which is incorporated by reference herein in its entirety), which is based on the Traveling Salesperson Problem (TSP). The mapping is done by employing a series of increasing recombination thresholds in order to minimalize the chance of mixing markers from different chromosome. By each step of increasing the recombination thresholds the shorter linkage groups have the potential to merge into a longer of the linkage groups.

A maximum likelihood estimate (MLE) for the presence of a marker is calculated, together with an MLE assuming no QTL effect, to avoid false positives. A login of an odds ratio (LOD) is then calculated as: LOD=log₁₀ (MLE for the presence of a QTL/MLE given no linked QTL). The LOD score essentially indicates how much more :likely the data are to have arisen assuming the presence of a QTL versus in its absence. The LOD threshold value for avoiding a. false positive with a given confidence, say 95%, depends on the number of markers and the length of the genome. Graphs indicating LOD thresholds are set forth in Lander and Botstein, Genetics, 121:185-199 (1989), and further described by Ares and Moreno-Gonzalez, Plant Breeding, Hayward, Bosemark Romagosa (eds.) Chapman & Hall, London, pp. 314-331 (1993). QTL prediction was done using MultiQTL program based on the linkage maps that were merged by Multipoint and the F2 population phenotype data. MuItiQTL use multiple interval mapping (MIM). MultiQTL. significance is computed with permutation, bootstrap tools and FDR for total analysis, as described in Korol et al. Enhanced Efficiency of Quantitative Trait Loci Mapping Analysis Based on Multivariate Complexes of Quantitative Traits, Genetics. 2001 April; 157(4): 1789-1803, which is incorporated by reference herein in its entirety.

Additional models can be used. Many modifications and alternative approaches to interval mapping have been reported, including the use of non-parametric methods (as described in Kruglyak and Lander, Genetics, 139:1421-1428 (1995), the entirety of which is incorporated by reference herein). Multiple regression methods or models also can be used, in which the trait is regressed on a large number of markers (as described in Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994), each of which is incorporated by reference herein in its entirety). Procedures combining interval mapping with regression analysis, whereby the phenotype is regressed onto a single putative QTL at a given marker interval, and at the same time onto a number of markers that serve as “cofactors,” have been reported by Jansen and Stam, Genetics, 136:1447-1455 (1994) and Zeng, Genetics, 136:1457-1468 (1994), each of which is incorporated by reference herein in its entirety. Generally, the use of cofactors reduces the bias and sampling error of the estimated. QTL positions (as described in Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), which is incorporated by reference herein in its entirety, thereby improving the precision and efficiency of QTL mapping (Zeng, Genetics, 136:1457-1468 (1994), each of which is incorporated by reference herein in its entirety). These models can be extended to multi-environment experiments to analyze genotype-environment interactions (as described in Jansen et al., Theo. Appl. Genet. 91:33-37 (1995), which is incorporated by reference herein in its entirety).

Selection of appropriate mapping populations is important to map construction. The choice of an appropriate mapping population depends on the type of marker systems employed (as described in Tanksley et al., Molecular mapping of plant chromosomes. chromosome structure and function: Impact of new concepts J. P. Gustafson and R. Appels (eds.), Plenum Press, New York, pp. 157-173 (1988), the entirety of which is incorporated by reference herein in its entirety). Consideration must be given to the source of parents (adapted vs. exotic) used in the mapping population. Chromosome pairing and recombination rates can be severely disturbed (suppressed) in wide crosses and generally yield greatly reduced linkage distances. Wide crosses will usually provide segregating populations with a relatively large array of polymorphisms when compared to progeny in a narrow cross.

An F2 population is the first generation of selfing after the hybrid seed is produced. In one example, a single F1 plant can be selfed to generate a population segregating for all the genes in Mendelian (1:2:1) fashion. Maximum genetic information is obtained from a completely classified F2 population using a codominant marker system (as described in Mather, Measurement of Linkage in Heredity: Methuen and Co., (1938), which is incorporated by reference herein in its entirety). In the case of dominant markers, progeny tests (e.g., F3, BCF2) are required to identify the heterozygotes, thus making it equivalent to a completely classified F2 population. However, this procedure is often prohibitive because of the cost and time involved in progeny testing. Progeny testing of F2 individuals is often used in map construction where phenotypes do not consistently reflect genotype (e.g., disease resistance) or where trait expression is controlled by a QTL. Segregation data from progeny test populations (e.g., F3 or BCF2) can be used in map construction. Marker-assisted selection can then be applied to cross progeny based on marker-trait map associations (F2, F3), where linkage groups have not been completely disassociated by recombination events (e.g., maximum disequilibrium).

Recombinant inbred lines (RIL) (genetically related lines; usually >F5, developed from continuously selfing F2 lines towards homozygosity) can be used as a mapping population. litifortnation obtained from dominant markers can be maximized by using RIL because all loci are homozygous or nearly so. Under conditions of tight linkage (e.g., about <10% recombination), dominant and co-dominant markers evaluated in RIL populations provide more information per individual than either marker type in backcross populations (as described in Reiter et at., Proc. Natl. Acad. Sci. (U.S.A.) 89:1477-1481 (1992), which is incorporated by reference herein in its entirety). However, as the distance between markers becomes larger (e.g., loci become more independent), the information in RIL populations decreases dramatically when compared to codominant markers.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus, a population is created consisting of individuals nearly like the recurrent parent but each individual carries varying amounts or a mosaic of genomic regions from the donor parent. Backcross populations can be useful for mapping dominant markers if all loci in the recurrent parent are homozygous and the donor and recurrent parent have contrasting polymorphic marker alleles (Reiter et at., Proc. Natl. Acad. Sci. (U.S.A.) 89:1477-1481 (1992)). Information obtained from backcross populations using either codominant or dominant markers is less than that obtained from F2 populations because one, rather than two, recombinant gametes are sampled per plant. Backcross populations, however. are more informative (at low marker saturation) when compared to RILs as the distance between linked loci increases in RIL populations (e.g., about 0.15% recombination). Increased recombination can be beneficial for resolution of tight linkages, but may be undesirable in the construction of maps with low marker saturation.

Near-isogenic lines (NIL) created by many backcrosses to produce an a av of individuals that are nearly identical in genetic composition except for the trait or genomic region under interrogation can be used as a mapping population. In mapping with NILs, only a portion of the polymorphic loci are expected to map to a selected region.

Bulk segregant analysis (BSA) is a method developed for the rapid identification of linkage between markers and traits of interest (as described in Michelmore, et at., Proc. Natl. Acad. Sci. (U.S.A.) 88:9828-9832 (1991), which is incorporated by reference herein in its entirety). In BSA, two bulked DNA samples are drawn from a segregating population originating from a single cross. These bulks contain individuals that are identical for a particular trait (resistant or susceptible to particular disease) or genomic region but arbitrary at unlinked regions (e.g., heterozygous). Regions unlinked to the target region will not differ between the bulked samples of many individuals in BSA.

An alternative to traditional QTL mapping involves achieving higher resolution by mapping haplotypes, versus individual markers (as described in Fan et at. 2006 Genetics), which is incorporated by reference herein in its entirety). This approach tracks blocks of DNA known as haplotypes, as defined by polymorphic markers, which are assumed to be identical by descent in the mapping population. This assumption results in a larger effective sample size, offering greater resolution of QTL. Methods for determining the statistical significance of a correlation between a phenotype and a genotype, in this case a haplotype, may be determined by any statistical test known in the art and with any accepted threshold of statistical significance being required. The application of particular methods and thresholds of significance are known in the art.

In another aspect, overlapping sets of clones can be derived by using the available markers of the present invention to screen, for example, BAC, PAC, bacteriophage P1, or cosmid libraries. In addition, hybridization approaches can be used to convert the YAC maps into BAC, PAC, bacteriophage P1, or cosmid contig maps. Entire YACs and products of inter-Alu-PCR as well as primer sequences from appropriate STSs can be used to screen BAC, PAC, bacteriophage P1, or cosmid libraries. The clones isolated for any region can be assembled into contigs using STS content information and fingerprinting approaches (as described in Su&ton et al., Comput. Appl. Biosci. 4:125-132 (1988)), which is incorporated by reference herein in its entirety.

According to another embodiment, the degeneracy of the genetic code, which allows different nucleic acid sequences to code for the same protein or peptide, is known in the literature. As used herein a nucleic acid molecule is degenerate of another nucleic acid molecule when the nucleic acid molecules encode for the same amino acid sequences but comprise different nucleotide sequences. An aspect of the present invention is that the nucleic acid molecules of the present invention include nucleic acid molecules that are degenerate of the nucleic acid molecule that encodes the protein(s) of the quantitative trait alleles.

Another aspect of the present invention is that the nucleic acid molecules of the present invention include nucleic acid molecules that are homologues of the nucleic acid molecule that encodes the one or more of the proteins associated with the QTL.

Exogenous genetic material may be transferred into a plant by the use of a DNA plant transformation vector or construct designed for such a purpose. A particularly preferred subgroup of exogenous material comprises a nucleic acid molecule of the present invention (as described in Plant Molecular Biology: A Laboratory Manual, eds. Clark, Springer, N.Y. (1997), which is incorporated by reference herein in its entirety).

In another aspect, a construct or vector may include the endogenous promoter of the scatter resistance QTL of the present invention. The characteristic of scatter resistance might best be achieved by expressing the identified QTL protein with the endogenous promoter. Alternatively, a heterologous promoter may be selected to express the protein or protein fragment of choice. These promoters may be operably linked to a polynucleotide sequence encoding the protein corresponding to the scatter resistance QTL. The heterologous promoter may be one that is selected based upon maturation or flowering time, in that timing of expression of the desired protein may be critical to the parameters affecting the scatter disease resistance trait. Effective expression of the scatter disease resistance QTL may require promoters that express in specific tissue types.

All patents and literature eferences cited in the present specification are hereby incorporated by reference herein in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to he included within, and can be made without departing from. the true scope of the invention.

### EXAMPLES

### Example 1

### QTL for Shattering-Resistant Sesame

This innovation presents a methodology of breeding sesame lines bearing shattering-resistant capsules. 99% of the sesame plants grown worldwide are harvested manually. The first and foremost obstacle to complete mechanization for this important crop is the dehiscence nature of its capsules. This innovative method includes the collection of worldwide sesame lines, the creation of F2 linkage populations, massive phenotyping and genotyping of thousands of sesame lines, prediction of QTLs affecting the shattering resistance trait, and the establishing of unique marker combinations (referred to herein as a “marker cassette”) for shattering resistant sesame lines never found before in commercial or natural lines.

Disclosed breeding methodology is based on the discovery of the Target Product Genomic Code (TPGC). The Target Product (TP) is defined in advance based on market requirements. It includes a set of desired attributes (traits) that are available in natural genetic variations and the Genomic Code (GC), which is a set of genomic regions that affect the Target Products' traits. Algorithms take the GC, which is composed of a quantitative trait locus (QTL) database linked to the TP, and define the Target Product Genomic Code (TPGC). The algorithms calculate multiple genomic interactions, including effects of heterosis and epistasis, and maximize the genomic potential of specific plants for the development of new varieties. The breeding program discovers the TPGC, then by crossing and selfing we progress until we achieve a product which contains the specific GC we discovered, A typical breeding project includes the following breeding and technical cycles:

Trait Discovery—where a broad spectrum of varieties from different geographies and worldwide sources are grown and phenotyped in order to discover new traits that can potentially be combined to create the new product.

Trait Blend—a crossing cycle based on phenotypic assumption, where we mix and combine the different traits. The initial trait cycle is followed by an additional cycle to create an F2 population, which will provide the basis for algorithmic analysis that will lead to the TPGC construction.

TPGC Discovery—the most important phase where every single plant is phenotyped and genotyped to produce a linkage map, discover the QTLs and discover the TPGC using our proprietary technology.

Line Validation 1.1—the first year of validating line version 1. These lines are based on millions of in silico selections and are defined as the project's pioneer varieties.

Line Validation 1.2—the second year of validating line version 1.

Pre-commercial 1.3—the third year and final validation of line version 1.′

Trait TPGC Blend—in this the phase we perform accurate crossing based on our proprietary algorithm, calculating the most efficient way to reach the best TPGC. The crossing is performed after in silico selection of millions of combinations. The trait TPGC blend phase is followed by an additional cycle to produce an F2 population for a second GC discovery. It is important to note that this phase is based on our algorithm, unlike the Trait Blend phase that is based on phenotype assumptions. Defining the TP for sesame includes identifying the shattering-resistant trait to enable harvesting mechanically. To identify the shattering-resistant capsules traits, a set of phenotype traits were developed to correlate with measured seed retention and capsule structure. The unique combination between the capsule structure and seed retention enable it to be harvested mechanically but still enable the seed to release easily by the thresher in the combine. For the unique combination, identifying a plurality of quantitative trait loci (“QTLs”) associated with it (GC) completes the TPGC for breeding sesame for mechanical harvesting.

The trait discovery is based on germplasm included five hundreds different sesame lines that were obtained from the U.S. National Plant Germplasm System (NPGC) and courtesy of Prof. Amram Ashri's sesame germplasm collection (Ashri, 1998). Screening for trait discovery was based on allocating traits related to capsule structure and capsule retention of the seeds.

150 different lines were produced for trait blend—crosses, executed based on the potential for enrichment of genomic diversity thus creating a new complex of traits for the shattering-resistant capsules as the initial step for a TP directed breeding program for shattering resistant sesame lines. The resulted F1 hybrids were later self-crossed to create F2 linkage populations that showed phenotypic segregation.

The F2 population then was planted in six (6) different environments for discovering the TPGC, including shattering resistant capsules traits. After screening 15000 individuals, a set of ˜3000 representatives (of F2) was selected. The selected individuals (the 3000 F2 plant) were massively phenotyped for three shattering-resistant capsule (SRC) components:

SRC1: An ordinal variable used to evaluate the rate of the seed retention in the field by shaking the plant and counting the amount seeds that have fallen to the ground, relative to the total number of seeds (those remaining inside the capsule plus those fallen). The SRC1 trait ranged from 1—denoting capsules losing all their seeds, to 10—denoting capsules retaining all their seeds.

SRC2: The rate (in %) of the seed retention, evaluated after the capsules are turned upside down calculated by counting the amount of the seeds that remain inside the capsules, divided by the total number of seeds (those remaining inside the capsule plus those fallen). The SRC2 trait ranged from 0, denoting all seeds were fallen to 100%, denoting all seed were remained inside the capsule.

SRC3: The ratio between the total length of the capsule (in mm) and the length of the zone in which the capsule tips are open (the abscission layer), calculated by measuring each of the lengths using a ruler. The SRC3 trait ranged from 0, denoting capsule is totally closed and 1, denoting the capsule is totally open.

All the shattering-resistant capsule trait's components were summarized into one representative trait which was named the shattering-resistant capsule trait. The selected ˜3000 individuals were genotyped under examination of a panel with 400 markers, based on single nucleotide polymorphism (SNP). These 400 markers' paid is directly designed based on parental lines' RNA sequences of each linkage F2. population. The panel was designed to maximize the chance to have the largest number of common segregate SNPs in order to create highly similar linkage maps for all observed populations.

### Mapping Population

The computation of linkage maps is executed on each linkage F2 population based on genotyping results. Linkage maps were computed with MultiPoint, an interactive package for ordering multi-locus genetic maps, and verification of maps based on resampling techniques.

### QTL Discovery

QTL discovery related to shattering resistance was executed with MultiQTL package. The program produced linkage maps that were merged by Multipoint and the F2 population phenotype data. MuitiQTL use multiple interval mapping (MIM). MultiQTL significance is computed with permutation, bootstrap tools and FDR for total analysis. The inventors have analyzed the linkage maps of all eight F2 populations and the information relating to the three shattering-resistant capsule traits over all genotyped plants that belong to the F2 populations, The prediction of QTL was in a “one trait to one marker” model, meaning that for all markers that constructed the linkage maps, each trait was tested independently against each one of the markers. The results point to 8 markers from 7 different linkage groups that are representing QTLs related to shattering resistance as described in Table 1. Each population presented a different marker-cassette related to shattering resistance but still some populations shared a subset of common markers with other populations. The varieties of marker-cassettes were summarized as described in FIGS. 1A, 1B and Tables 1 and 2 below.

### Significance and Co-Occurrences of Shattering Resistant Capsules Markers

The QTL, analysis provided the set of markers that represent QTL related to shattering resistant capsules in sesame for each linkage F2 population separately. In order to strengthen the significance of each marker, an in-house algorithm was developed to observe genotype-phase of each marker related to QTL/trait in all linkage F2 populations in different environments. The occurrence of shattering resistance capsules marker in two or more linkage F2 population (repetitive markers) strengthen its significance as representative for shattering resistant capsules QTL. In addition, the co-occurrence of non-repetitive and repetitive markers related to shattering resistance capsules in a given population was observed for the design of “marker-cassettes” that provides the genetic signature for shattering resistant capsules in sesame lines.

### In-Silico Self- and Cross-Self Based Breeding Program

Based on the QTL prediction, which provide the effect of each phase of a given marker for each of the three shattering-resistant capsule traits, three different algorithms for the simulation and prediction of the genotypic state of self, cross-self and hybrid plant was in-house developed for processing the TPGC blend. The TPGC blend combines QTLs from different populations together into a single plant to increase similarity of the discovered TPGC to an existing product, which contains a unique cassette of QTLs for shattering-resistant capsules which never existed before. The algorithms design in silico millions of selfing combination from F2 to F8, millions of new combinations of F1 and then selfing to F8 and millions of F1 hybrids to create hybrid variety. This, in order to measure the potential for each of the 3000 plants to acquire the shattering-resistant capsules in the right combination at the right phase. After running the analysis among ˜3000 plants, 200 higher score plants were chosen for the selling, cross selfing and hybrid programs.

### Validation of Shattering-Resistant Capsules Lines

After the determination of which plants have the highest potential to acquire shattering resistant capsules based on genetic code, it is important to preserve this potential in next generations. In order to follow the genetic code of the shattering resistant capsules “marker cassettes”, the offspring of each chosen lines (the next generation) were genotyped based on the shattering resistant capsules “marker cassettes”. Only offspring that present the previous generation's “marker cassette” for shattering resistant capsules were selected and forwarded to the next generation. This procedure ensures the maintenance of the shattering resistant capsules trait and “marker-cassette” for each shattering resistant line. This invention presents a methodology for the design of four marker-cassettes that point, with one marker cassette or more, to shattering resistant capsules sesame lines, as follows:

In one embodiment, marker cassette 1 comprises the genetic markers LG3_19205572, LG7_5141423, LG15_5315334, or a combination thereof. In some embodiments the alleles for said LG3_19205572, LG7_5141423, and LG15_5315334 are homozygous or heterozygous. In one embodiment, marker cassette 2 comprises: LG3_19205572, LG11_8864255, LG15_5315334, or a combination thereof. In some embodiments the alleles for said L3_19205572, LG11_8864255, and LG15 _5315334 are homozygous or heterozygous. In one embodiment, marker cassette 3 comprises LG3_19205572, LG5_12832234, LG15_4900868, LG15_5315334, or a combination thereof. In some embodiments the alleles for said LG3_19205572, LG5_12832234, LG15_4900868, and LG15_5315334 are homozygous or heterozygous.

In one embodiment, marker cassette 4 comprises LG6_2739268, LG11_8864255, LG16 _1563304, or combination thereof. In some embodiments the alleles for said LG6_2739268, LG11_8864255, and LG16_1563304 are homozygous or heterozygous.

Tables 1 and 2 summarize the genetic markers, QTLs, corresponding phenotypic traits (SRC1, 2 and 3 defined above) and significance values, as well as the characterization of the QTL (marker) cassettes having the shattering-resistant traits. The reference alleles are based on the Sesamum indicum reference Genome V1.0 (as described in Wang L, Yu S, Tong C, et al. Genome sequencing of the high oil crop sesame provides insight into oil biosynthesis. Genome biology, 2014, 15(2): R39), which is incorporated by reference herein in its entirety, as indicated in U.S. Pat. No. 10,577,623 and in FIG. 1A with reference to linkage group (LG) number; and are also indicated with respect to the updated Sesamum indicum reference Genome V2.0 genetic map (as described in Wang et al. 2016, Updated sesame genome assembly and fine mapping of plant height and seed coat color QTLs using a new high-density, BMC Genomics 17:31, which is incorporated by reference herein in its entirety), as illustrated in FIG. 1B with reference to chromosome number and strand location (indicated by “+” for the reference strand and by “−” for the reverse complement strand). It is further noted that the two markers referred to as QTL 6 in U.S. Pat. 10,577,623 are indicated herein as QTLs 6 and 7, as they have been identified as being unlinked and therefore independent of each other, and QTL 7 has been renumbered QTL 8. The SEQ ID NOs were left unchanged.

The QTL p-values are the significance level of single-QTL analysis computed by the MultiQTL program.

TABLE 1
QTLs, genetic markers, corresponding phenotypic traits and significance
values.

| | SEQ | Marker name | | | QTL |
|---|---|---|---|---|---|
| | ID | (LG/Chromosome and position) | | Phenotypic | P- |
| QTL | NO | Genome V1.0 | Genome V2.0 | trait | value |
| 1 | 1, 9 | LG3_19205572 | (3+, 20792058) | SRC3 | 0.05 |
| | | (3, 19205572) | | | |
| 2 | 2, 10 | LG5_12832234 | (5−, 3973879) | SRC3 | 0.025 |
| | | (5, 12832234) | | | |
| 3 | 3, 11 | LG6_2739268 | (6−, 22739924) | SRC3 | 0.045 |
| | | (6, 2739268) | | | |
| 4 | 4, 12 | LG7_5141423 | (7+, 8855456) | SRC1, SRC3 | 0.0075 |
| | | (7, 5141423) | | | |
| 5 | 5, 13 | LG11_8864255) | (11+, 9582098) | SRC3 | 0.003 |
| | | (11, 8864255) | | | |
| 6 | 6, 14 | LG15_4900868 | (9−, 17947299) | SRC1, SRC2, | 0.0005 |
| | | (15, 4900868) | | SRC3 | |
| 7 | 7, 15 | LG15_5315334 | (9−, 17532833) | SRC1, SRC2, | 0.0005 |
| | | (15, 5315334) | | SRC3 | |
| 8 | 8, 16 | LG16_1563304 | (12+, 1563304) | SRC3 | 0.038 |
| | | (16, 1563304) | | | |

TABLE 2
Characterization of the QTL cassettes.

| | SEQ | Allele | | Cassette (with respective QTLs) | | | |
|---|---|---|---|---|---|---|---|
| QTL | ID NO | 1 (ref) | 2 | 1 | 2 | 3 | 4 |
| 1 | 1, 9 | C | T | CC/CT | CC/CT | CC/CT | . |
| 2 | 2, 10 | C | T | . | . | CC/CT | . |
| 3 | 3, 11 | T | C | . | . | . | CC/CT |
| 4 | 4, 12 | C | G | CC/CG | . | . | . |
| 5 | 5, 13 | C | G | . | CC/CG | . | CC/CG |
| 6 | 6, 14 | G | A | . | . | AA/AG | . |
| 7 | 7, 15 | T | C | CC/CT | CC/CT | CC/CT | . |
| 8 | 8, 16 | A | G | . | . | . | GG/AG |

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What ts clamed is:

1. A sesame plant having shatter-resistant capsules, progeny thereof and/or part(s) thereof, wherein the sesame plant comprises at least three shattering-resistant capsule quantitative trait loci (QTLs) having corresponding nucleic acid genetic markers that are associated with seed retention phenotypic traits of the sesame plant, wherein the QTLs are combined in the sesame plant from a plurality of sesame varieties by computationally supported breeding, and comprise at least three of:
QT1 with corresponding marker set forth in SEQ ID NO: 1 or 9, wherein e sesame plant is homozygous with respect to SEQ ID NO: 1 or heterozygous at QTL 1,
QTL 2 with corresponding marker set forth in SEQ ID NO: 2 or 10, wherein the sesame plant is homozygous with respect to SEQ ID NO: 2 or heterozygous at QTL 2,
QTL 3 with corresponding marker set forth in SEQ ID NO: 3 or 11, wherein the sesame plant is homozygous with respect to SEQ ID NO: 11 or heterozygous at QTL 3,
QTL 4 with corresponding marker set forth in SEQ ID NO: 4 or 12, wherein the sesame plant is homozygous with respect to SEQ ID NO: 4 or heterozygous at QTL 4,
QTL 5 with corresponding marker set forth in SEQ ID NO: 5 or 13, wherein the sesame plant is homozygous with respect to SEQ ID NO: 5 or heterozygous at QTL 5,
QTL 6 with corresponding marker set forth in SEQ ID NO: 6 or 14, wherein the sesame plant is homozygous with respect to SEQ ID NO: 14 or heterozygous at QTL 6,
QTL 7 with corresponding marker set forth in SEQ ID NO: 7 or 15, wherein the sesame plant is homozygous with respect to SEQ ID NO: 15 or heterozygous at QTL 7, and
QTL 8 with corresponding marker set forth in SEQ ID NO: 8 or 16, wherein the sesame plant is homozygous with respect to SEQ ID NO: 16 or heterozygous at QTL 8.

2. The sesame plant having shatter-resistant capsules, progeny thereof and/or parts) thereof according to claim 1, wherein the QTLs comprise:
QTL 1 with corresponding marker set forth in SEQ ID NO: 1 or 9, wherein e sesame plant is homozygous with respect to SEQ ID NO: 1 or heterozygous at QTL 1,
QTL 2 with corresponding marker set forth in SEQ ID NO: 2 or 10, wherein the sesame plant is homozygous with respect to SEQ ID NO: 2 or heterozygous at QTL 2, and
QTL 6 with corresponding marker set forth in SEQ ID NO: 6 or 14, wherein the sesame plant is homozygous with respect to SEQ ID NO: 14 or heterozygous at QTL 6, and
QTL 7 with corresponding marker forth in SEQ ID NO: 7 or 15, wherein the sesame plant is homozygous with respect to SEQ ID NO: 15 or heterozygous at QTL 7.

3. The sesame plant having shatter-resistant capsules, progeny thereof and/or part(s) thereof according to claim 1, wherein the QTLs comprise:
QTL 1 with corresponding marker set forth in SEQ ID NO: 1 or 9, wherein the sesame plant is homozygous with respect to SEQ ID NO: 1 or heterozygous at QTL1,
QTL 4 with corresponding marker set forth in SEQ ID NO: 4 or 12, wherein the sesame plant is homozygous with respect to SEQ ID NO: 4 or heterozygous at QTL 4, and
QTL 7 with corresponding marker set forth in SEQ ID NO: 7 or 15, wherein the sesame plant is homozygous with respect to SEQ ID NO: 15 or heterozygous at QTL 7.

4. The sesame plant having shatter-resistant capsules, progeny thereof and/or part(s) thereof according to claim 1, wherein the shattering-resistant capsules are fully or partly shattering-resistant capsules, wherein shatter-resistant capsules are measured in fully developed capsules having at most 10% seed moisture, and wherein the shatter-resistant capsules have at least one of:
at least 80% seed retention after shaking the plant,
at least 80% seed retention after the capsules are turned upside down,
a ratio of at least 5:1 between a total :length of the capsule and alength of a zone in which the capsule tips are open,
20-30% of the capsules retain 90-95% of the seeds in fully developed green capsules before drying.

5. The sesame plant having shatter-resistant capsules, progeny thereof and/or part(s) thereof according to claim 1, wherein the plant is a hybrid and/or the part thereof is any of: a seed, an endosperm, an ovule, pollen, cell, cell culture, tissue culture, plant organ, protoplast, meristem, embryo, or a combination thereof.

6. A sesame plant having shatter-resistant capsules, progeny thereof and/or part(s) thereof, wherein the sesame plant comprises a plurality of shattering-resistant capsule quantitative trait loci (QTLs) having corresponding nucleic acid genetic markers that are associated with seed retention phenotypic traits of the sesame plant, wherein the QTLs are combined in the sesame plant from a plurality of sesame varieties by computationally supported breeding, and comprise:
QTL 1 with corresponding marker set forth in SEQ ID NO: 1 or 9, wherein the sesame plant is homozygous with respect to SEQ ID NO: 1 or heterozygous at QTL 1,
QTL 2 with corresponding marker set forth in SEQ ID NO: 2 or 10, wherein the sesame plant is homozygous with respect to SEQ ID NO: 2 or heterozygous at QTL 2,
QTL 6 with corresponding marker set forth in SE.Q ID NO: 6 or 14, wherein the sesame plant is homozygous with respect to SEQ ID NO: 14 or heterozygous at QTL 6, and
QTL 7 with corresponding marker set forth in SEQ ID NO: 7 or 15, wherein the sesame plant is homozygous with respect to SEQ ID NO: 15 or heterozygous at QTL 7.

7. The sesame plant having shatter-resistant capsules, progeny thereof and/or part(s) thereof according to claim 6, wherein the shattering-resistant capsules are fully or partly shattering-resistant capsules, wherein shatter-resistant capsules are measured in fully developed capsules having at most 10% seed moisture, and wherein the shatter-resistant capsules have at least one of:
at least 80% seed retention after shaking the plant,
at least 80% seed retention after the capsules are turned upside down,
a ratio of at least 5:1 between a total length of the capsule and a length of a zone in whichthe capsule tips are open,
20-30% of the capsules retain 90-95% of the seeds in fully developed green capsules before drying.

8. The sesame plant having shatter-resistant capsules, progeny thereof and/or part(s) thereof according to claim 6, wherein the plant is a hybrid and/or the part thereof is any of: a seed, an endosperm, an ovule, pollen, cell, cell culture, tissue culture, plant organ, protoplast, meristem, embryo, or a combination thereof.

9. A sesame plant having shatter-resistant capsules, progeny thereof and/or part(s) thereof, wherein the sesame plant comprises a plurality of shattering-resistant capsule quantitative trait loci (QTLs) having corresponding nucleic acid genetic markers that are associated with seed retention phenotypic traits of the sesame plant, wherein the QTLs are combined in the sesame plant from a plurality of sesame varieties by computationally supported breeding, and comprise:
QTL 1 with corresponding marker set forth in SEQ ID NO: 1 or 9, wherein the sesame plant is homozygous with respect to SEQ ID NO: 1 or heterozygous at QTL1,
QTL 4 with corresponding marker set forth in SEQ ID NO: 4 or 12, wherein the sesame plant is homozygous with respect to SEQ ID NO: 4 or heterozygous at QTL 4, and
QTL 7 with corresponding marker set forth in SEQ ID NO: 7 or 15, wherein the sesame plant is homozygous with respect to SEQ ID NO: 15 or heterozygous at QTL 7.

10. The sesame plant having shatter-resistant capsules, progeny thereof and/or pants) thereof according to claim 9, wherein the shattering-resistant capsules are fully or partly shattering-resistant capsules, wherein shatter-resistant capsules are measured in fully developed capsules having at most 10% seed moisture, and wherein the shatter-resistant capsules have at least one of:
at least 80% seed retention after shaking the plant,
at least 80% seed retention after the capsules are turned upside down,
a ratio of at least 5:1 between a total length of the capsule and a length of a zone in which the capsule tips are open,
20-30% of the capsules retain 90-95% of the seeds in fully developed green capsules before drying.

11. The sesame plant having shatter-resistant capsules, progeny thereof and/or part(s) thereof according to claim 9, wherein the plant is a hybrid and/or the part thereof is any of: a seed, an endosperm, an ovule, pollen, cell, cell culture, tissue culture, plant organ, protoplast, meristem, embryo, or a combination thereof.


SEQUENCE LISTING (16 sequences)

SEQ ID NO: 1 - DNA, 301 bp, Sesamum indicum
cctttagcagggcatcactctcttcaacatcgtactgcaccgagaggaatttcgaagtgagaaaaacttggggctccgacacttcccctttgctgtcgttttgaaaagtgagcttcaatttcgccaaattatccaacaatttgcacgagactttattaaagaacacggagctctcgctgtcgaactccgacgtaactcgcagcttcggccgacccatttggaagagccccagaaaaccgccgcctttcgaaccgccgtcgtccacggcgggggatggtggtggagacagataatagggttg

SEQ ID NO: 2 - DNA, 301 bp, Sesamum indicum
ccgggccacaactgatgcttattgtgttgccaaatatggtcagaagtgggtcaggacaaggacaatcatcgacagttttgctcctaaatggaatgagcagtatacttgggaagtttttgatccttgcactgttgtcaccattggtgtattcgataattgtcatctgcaaggtggagataaagctggaagggattcaagaattgggaaggtaagaattcgcctttcaactctggaaacagaccgtgtgtacactcattcttatcctcttctagttttgcatccttccggggtaaaaaagatg

SEQ ID NO: 3 - DNA, 301 bp, Sesamum indicum
ttagggcgtcgaattacttggacgcgccgagcaagctagcctgctgcaactcaagctcgttgagctgctgctcgcggtccatctctataatcagaggcacgacgagcacgaggaaggtggtgccggcaatccacgcggcttttccggtgctcttcaggagcttctttgcgacgtaggcggtatcaaaagccgctttcttgccgcggtacacgatgggtgactgggaaacggaggtggaaacacgggacaggattccgtcgtccgacgacgctccgcctcctctagtagacattctggtgaa

SEQ ID NO: 4 - DNA, 301 bp, Sesamum indicum
gccattgatttaccaaaagcaccaacccattttggtgagataattgggaaacttgttttggctggagccttggacttcaacaaggtggcaagggatattctggcaaaagtaggtgacgactattaccaaaaggccatatttactgctgctctgaaggttgtcagctctgatccttcaggaaaggcattgctcgattcacaggcgtctgatgtcgctgcctgcgagagtttattttagagctcactccttgttatgggaattactggaaacatttgtaacctcatagaagaaatgtgctatt

SEQ ID NO: 5 - DNA, 301 bp, Sesamum indicum
aatagaaaattggtaattcaaaagggaaagatggaaaaatgattaccatccctgtcttcagctgcctcaacattcacagaatccttcgcaagcacatctgtctctgtggatgctttactttttattaccggaaactgaacattgtgccgtccatttttcctaaaaagcatataatctctctttgacagtgttttcattacaagagttcctgcatttccatcattctaagaaagagaggttgattaaggcatccagcatcgcataaacataaccaggaaaatcgggagcaaacatagtactg

SEQ ID NO: 6 - DNA, 301 bp, Sesamum indicum
ggaggcaaaagaatacgggttggttgatgcagtgatcgatgatggcaagcctggactagtcgcacccatcgcagatactgcacccccaccaaaaacccgtgtctgggatctttggaaaatcgaaggcagtaaaaaagccaagaaaaacttgccctccgaagagaaactattacaaaatggatacacagttggccaaggtgaagatgacagaagcacggaacaggtagaggaagcaccaacatctcaatgagtaatgaatgttgagatatttcttgtatacactgtcaaacattgtagctag

SEQ ID NO: 7 - DNA, 301 bp, Sesamum indicum
agttgataaactgttgactaatcaaataatacgcattctgcacgcactcacaaatactatgattgttgtttactgaataaggttttcatggaattttcacaggttaaattctagtaatcacataaaagtatgtcgccagctgactcttcatgcgaggaaaatgtgtacatggccaagttggccgaacaggctgagaggtatgaggagatggttgaattcatggagaaggttgtgaaggccgtggacactgatgagctgacagtcgaggaaaggaaccttctctctgtggcatacaagaatg

SEQ ID NO: 8 - DNA, 301 bp, Sesamum indicum
acgtaaatgtttggatttaatgtaatttaatctaatgatattgcaaatgagtaaattactcccaaattattcggataaagcaatttaacttttggtttcttgtgagataacattgcatgtcctttatgaaccaagagcagtggccgagggactggtggtggtaccgttgccaaggatgcattaggcaatgatgttattgcagcggaatggctcaaaaaccatggacctggcgatcggacacttacacaggggctgaaggtaattattgatctagttgcaaaatagatcacttattggcttt

SEQ ID NO: 9 - DNA, 301 bp, Sesamum indicum
cctttagcagggcatcactctcttcaacatcgtactgcaccgagaggaatttcgaagtgagaaaaacttggggctccgacacttcccctttgctgtcgttttgaaaagtgagcttcaatttcgccaaattatccaacaatttgcacgagattttattaaagaacacggagctctcgctgtcgaactccgacgtaactcgcagcttcggccgacccatttggaagagccccagaaaaccgccgcctttcgaaccgccgtcgtccacggcgggggatggtggtggagacagataatagggttg

SEQ ID NO: 10 - DNA, 301 bp, Sesamum indicum
ccgggccacaactgatgcttattgtgttgccaaatatggtcagaagtgggtcaggacaaggacaatcatcgacagttttgctcctaaatggaatgagcagtatacttgggaagtttttgatccttgcactgttgtcaccattggtgtatttgataattgtcatctgcaaggtggagataaagctggaagggattcaagaattgggaaggtaagaattcgcctttcaactctggaaacagaccgtgtgtacactcattcttatcctcttctagttttgcatccttccggggtaaaaaagatg

SEQ ID NO: 11 - DNA, 301 bp, Sesamum indicum
ttagggcgtcgaattacttggacgcgccgagcaagctagcctgctgcaactcaagctcgttgagctgctgctcgcggtccatctctataatcagaggcacgacgagcacgaggaaggtggtgccggcaatccacgcggcttttccggtgcccttcaggagcttctttgcgacgtaggcggtatcaaaagccgctttcttgccgcggtacacgatgggtgactgggaaacggaggtggaaacacgggacaggattccgtcgtccgacgacgctccgcctcctctagtagacattctggtgaa

SEQ ID NO: 12 - DNA, 301 bp, Sesamum indicum
gccattgatttaccaaaagcaccaacccattttggtgagataattgggaaacttgttttggctggagccttggacttcaacaaggtggcaagggatattctggcaaaagtaggtgacgactattaccaaaaggccatatttactgctgctgtgaaggttgtcagctctgatccttcaggaaaggcattgctcgattcacaggcgtctgatgtcgctgcctgcgagagtttattttagagctcactccttgttatgggaattactggaaacatttgtaacctcatagaagaaatgtgctatt

SEQ ID NO: 13 - DNA, 301 bp, Sesamum indicum
aatagaaaattggtaattcaaaagggaaagatggaaaaatgattaccatccctgtcttcagctgcctcaacattcacagaatccttcgcaagcacatctgtctctgtggatgctttactttttattaccggaaactgaacattgtgccgtgcatttttcctaaaaagcatataatctctctttgacagtgttttcattacaagagttcctgcatttccatcattctaagaaagagaggttgattaaggcatccagcatcgcataaacataaccaggaaaatcgggagcaaacatagtactg

SEQ ID NO: 14 - DNA, 301 bp, Sesamum indicum
ggaggcaaaagaatacgggttggttgatgcagtgatcgatgatggcaagcctggactagtcgcacccatcgcagatactgcacccccaccaaaaacccgtgtctgggatctttggaaaatcgaaggcagtaaaaaagccaagaaaaacttaccctccgaagagaaactattacaaaatggatacacagttggccaaggtgaagatgacagaagcacggaacaggtagaggaagcaccaacatctcaatgagtaatgaatgttgagatatttcttgtatacactgtcaaacattgtagctag

SEQ ID NO: 15 - DNA, 301 bp, Sesamum indicum
agttgataaactgttgactaatcaaataatacgcattctgcacgcactcacaaatactatgattgttgtttactgaataaggttttcatggaattttcacaggttaaattctagtaatcacataaaagtatgtcgccagctgactcttcacgcgaggaaaatgtgtacatggccaagttggccgaacaggctgagaggtatgaggagatggttgaattcatggagaaggttgtgaaggccgtggacactgatgagctgacagtcgaggaaaggaaccttctctctgtggcatacaagaatg

SEQ ID NO: 16 - DNA, 301 bp, Sesamum indicum
acgtaaatgtttggatttaatgtaatttaatctaatgatattgcaaatgagtaaattactcccaaattattcggataaagcaatttaacttttggtttcttgtgagataacattgcatgtcctttatgaaccaagagcagtggccgaggggctggtggtggtaccgttgccaaggatgcattaggcaatgatgttattgcagcggaatggctcaaaaaccatggacctggcgatcggacacttacacaggggctgaaggtaattattgatctagttgcaaaatagatcacttattggcttt